(19) United States
(12) Patent Application Publication
(10) Pub. No.: US 2023/0165745 A1
(43) Pub. Date: Jun. 1, 2023

(54) MASSAGE DEVICE CONTROLLING THE OPERATION OF THE ARM MASSAGE UNIT THROUGH ELECTRODES

(71) Applicant: BODYFRIEND CO., LTD., Seoul (KR)

(72) Inventors: Deok Hyun KONG, Seoul (KR); Jong Sick PARK, Seoul (KR); Sung Gu LIM, Seoul (KR)

(21) Appl. No.: 18/071,541

(22) Filed: Nov. 29, 2022

(30) Foreign Application Priority Data
Nov. 30, 2021 (KR) 10-2021-0169550
Jan. 14, 2022 (KR) 10-2022-0006099

(51) Int. Cl. A61H 9/00

(52) U.S. Cl. CPC A61H 9/0078; A61H 2201/1635; A61H 2205/06; A61H 2201/5058; A61H 2201/5071; A61H 2201/0149; A61H 2203/0437; A61H 2201/5012; A61H 2201/5097; A61H 2201/1207; A61H 2201/164; A61H 2205/106; A61H 2205/12; A61H 2201/5048; A61H 2230/065; A61H 2201/10; A61H 2201/0207; A61N 5/0616

(57) ABSTRACT

Provided herein is a massage apparatus including: an arm massage unit for massaging a user's arm; and a control unit for controlling the arm massage unit. The arm massage unit comprises a space and an electrode arranged in the space, and the control unit determines whether an electrical value of the electrode is changed, and controls the operation of the arm massage unit based on a determination result and an operation state of the arm massage unit.

### BACKGROUND OF THE INVENTION

### Field of the Invention

The present disclosure relates to a massage apparatus controlling operation of an arm massage unit through electrodes.

### Background Art

Massage is a medical adjuvant therapy for controlling the modulation of the body of a user helping blood circulation, and relieving fatigue of the user by applying various types of mechanical stimulation to a portion of the body, such as massaging, pressing, pulling, tapping, or moving a portion of the body of the user, helping blood circulation.

The growth in a massage demand caused an increase in demand for massage apparatuses or massage devices, which provide an artificial massage function, because of economic feasibility and temporal reasons. That is, as the demand for relieving fatigue or stress is increased while loosening the agglomerated muscles through massage, various mechanical massage apparatuses for time and cost efficiencies are being released. Any type of instruments, devices, or apparatuses for performing massage without a massagist is referred to as a massage apparatus.

Furthermore, demand for a massage apparatus capable of relieving fatigue or stress and promoting health through massage is increasing.

Therefore, studies for providing a massage apparatus capable of promoting health of users have continued.

Korean Patent No. 10-0941919 discloses a massage apparatus for providing massage capable of promoting a user's health.

### SUMMARY OF THE INVENTION

A massage apparatus according to the present disclosure can sense a user's arm to control motions of an arm massage unit which massages the user's arm.

In addition, a program for implementing an operating method of the massage apparatus described above may be recorded in a computer-readable recording medium.

According to the present disclosure, there is provided a massage apparatus including: an arm massage unit for massaging a user's arm; and a control unit for controlling the arm massage unit, wherein the arm massage unit comprises a space and an electrode arranged in the space, and the control unit determines whether an electrical value of the electrode is changed, and controls the operation of the arm massage unit based on a determination result and an operation state of the arm massage unit.

In addition, a program for implementing an operating method of the massage apparatus described above may be recorded in a computer-readable recording medium.

The massage apparatus according to an embodiment of the present disclosure controls the operation of the arm massage unit by sensing the user's arm, thereby preventing power waste of the arm massage unit and preventing a problem that user satisfaction with the massage apparatus gets lower.

The effects of the present disclosure are not limited to the above-mentioned effects, and other effects, which are not specifically mentioned herein, will be clearly understood by those skilled in the art from the following description.

### BRIEF DESCRIPTION OF THE DRAWINGS

FIG. 1 is a view for depicting a massage apparatus according to an embodiment of the present disclosure.

FIG. 2 is a view for depicting a main frame according to an embodiment of the present disclosure.

FIG. 3 is a view illustrating a massage apparatus according to an embodiment of the present disclosure.

FIG. 4 is a view illustrating an external device capable of communicating with a massage apparatus according to an embodiment of the present disclosure.

FIG. 5 is a view for depicting a first bio-signal measuring unit according to an embodiment of the present disclosure.

FIG. 6 is a view for depicting a second bio-signal measuring unit according to an embodiment of the present disclosure.

FIG. 7 is a view for depicting a method of measuring body composition by a user through the second bio-signal measuring unit according to an embodiment of the present disclosure.

FIG. 8 is a perspective view of a first bio-signal measuring unit according to an embodiment of the present disclosure.

FIG. 9 is a view illustrating components included in the first bio-signal measuring unit according to an embodiment of the present disclosure.

FIG. 10 is a view illustrating a first bio-signal measuring unit according to an embodiment of the present disclosure.

FIG. 12 is a view illustrating a second bio-signal measuring unit according to an embodiment of the present disclosure.

FIG. 13 is a view illustrating a second bio-signal measuring unit according to an embodiment of the present disclosure.

FIG. 14 is a view illustrating a second bio-signal measuring unit according to an embodiment of the present disclosure.

FIG. 15 is a view for depicting an example that a posture of the massage apparatus is adjusted to measure a bio-signal according to an embodiment of the present disclosure.

FIG. 16 is a view for depicting a first bio-signal measuring unit according to an embodiment of the present disclosure.

FIG. 17 is a view illustrating a magnet according to an embodiment of the present disclosure.

FIG. 18 is a perspective view of a first bio-signal measuring unit according to an embodiment of the present disclosure.

FIG. 19 is a view illustrating components included in the first bio-signal measuring unit according to an embodiment of the present disclosure.

FIG. 20 is a view illustrating a first bio-signal measuring unit according to an embodiment of the present disclosure.

FIG. 21 is a view illustrating a first bio-signal measuring unit according to an embodiment of the present disclosure.

FIG. 22 is a view illustrating a third bio-signal measuring unit according to an embodiment of the present disclosure.

FIG. 23 is a view illustrating a second bio-signal measuring unit and a fourth bio-signal measuring unit according to an embodiment of the present disclosure.

FIG. 24 is a view illustrating a fourth bio-signal measuring unit according to an embodiment of the present disclosure.

FIG. 25 is a view illustrating a second bio-signal measuring unit according to an embodiment of the present disclosure.

FIG. 26 is a view for depicting functions of a functional button according to an embodiment of the present disclosure.

FIG. 27 is a view for depicting an arm massage unit according to an embodiment of the present disclosure.

FIG. 28 is a view for depicting an arm air cell according to an embodiment of the present disclosure.

FIGS. 29A-29B are views illustrating a sensing unit according to an embodiment of the present disclosure, wherein FIG. 29A illustrates an example for depicting a position of the sensing unit, and FIG. 29B illustrates an example of the sensing unit according to an embodiment of the present disclosure.

FIGS. 30A-30B are views for depicting a state of an arm air cell according to an embodiment of the present disclosure. Specifically, FIG. 30A is a view for depicting a contracted state of the arm air cell according to an embodiment of the present disclosure, and FIG. 30B is a view for depicting an expanded state of the arm air cell according to an embodiment of the present disclosure.

FIG. 31 is a view for depicting an example of an arm massage unit control operation of a control unit according to an embodiment of the present disclosure.

FIG. 32 is a view for depicting an example of the arm massage unit control operation illustrated in FIG. 31.

FIG. 33 is a view for depicting another example of an arm massage unit control operation of a control unit according to an embodiment of the present disclosure.

FIG. 34 is a view for depicting an example of the arm massage unit control operation illustrated in FIG. 32.

FIG. 35 is a view for depicting a further example of an arm massage unit control operation of a control unit according to an embodiment of the present disclosure.

FIG. 36 is a view for depicting an example of the arm massage unit control operation illustrated in FIG. 35.

FIG. 37 is a view for depicting operations of an arm massage unit and a body massage unit according to an embodiment of the present disclosure.

### DETAILED DESCRIPTION OF THE PREFERRED EMBODIMENT

Advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art can fully understand the scope of the present disclosure.

Terms used herein will be briefly described, and the disclosed embodiments will be described in detail.

The terms including descriptive or technical terms which are used herein should be construed as having meanings that are obvious to one of ordinary skill in the art. However, the terms may have different meanings according to an intention of one of ordinary skill in the art, precedent cases, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

The singular forms of the components may be understood into the plural forms unless otherwise specifically stated in the context. Furthermore, the plural forms may include singular forms unless otherwise specifically stated in the context.

In the entire specification of the present disclosure, when a certain part “includes” a certain component, other components are not excluded unless explicitly described otherwise, and other components may in fact be included.

The term, “unit”, used in the present disclosure means a software element or a hardware element, and the “unit” performs some roles. However, the term, “unit”, is not limited to software or hardware. The “unit” may be configured in an addressable storage medium or may be configured to play one or more processors. Therefore, as an example, a “unit” includes elements, such as software elements, object-oriented software elements, class elements, and task elements, processes, functions, attributes, procedures, subroutines, segments of program codes, drivers, firmware, microcode, circuits, data, databases, data structures, tables, arrays, and variables. Functions provided within the elements and “units” may be combined with a smaller number of elements and “units” or may be further divided into additional elements and “units”.

According to an embodiment of the present disclosure, the “unit” may be implemented as a processor and a memory. The term “processor” should be interpreted broadly to encompass a general-purpose processor, a central processing unit (CPU), a microprocessor, a digital signal processor (DSP), a controller, a microcontroller, a state machine, and the like. In some environments, a “processor” may refer to an application specific integrated circuit (ASIC), a programmable logic device (PLD), a field programmable gate array (FPGA), or the like. The term “processor” may refer to a combination of processing devices, such as a combination of a DSP genome and a microprocessor, a combination of a plurality of microprocessors, a combination of one or more microprocessors in combination with a DSP couple core, or a combination of any other such configurations.

The term “memory” should be interpreted broadly to include any electronic component capable of storing electronic information. The term memory may refer to various types of processor-readable media such as random access memory (RAM), read-only memory (ROM), non-volatile random access memory (NVRAM), programmable read-only memory (PROM), erase-programmable read-only memory (EPROM ACL), electrically erasable PROM (EEPROM), flash memory, magnetic or optical data storage, registers, and the like. Memory is referred to as being in electronic communication with the processor if the processor can read information from, and/or write information to, the memory. The memory integrated in the processor is in electronic communication with the processor.

In the present specification, an actuator refers to a configuration capable of providing a driving force. For example, the actuator may include a motor, a linear motor, an electronic motor, a DC motor, an AC motor, a linear actuator, an electric actuator, and the like, but is not limited thereto.

In the present specification, according to an embodiment, a massage apparatus may refer to a massage apparatus including at least one massage unit among a body massage unit, an arm massage unit and a leg massage unit. In addition, according to another embodiment, a body massage unit, an arm massage unit and a leg massage unit may be provided as separate devices (for example, a body massage apparatus, an arm massage apparatus, and a leg massage apparatus), and the massage apparatus may refer to at least one massage unit among a body massage unit, an arm massage unit and a leg massage unit.

Hereinafter, embodiments of the present disclosure will be described in detail with reference to the accompanying drawings so as to be easily carried out by a person skilled in the art to which the present disclosure pertains. In addition, in order to clearly describe the present disclosure, portions irrelevant to the description will be omitted.

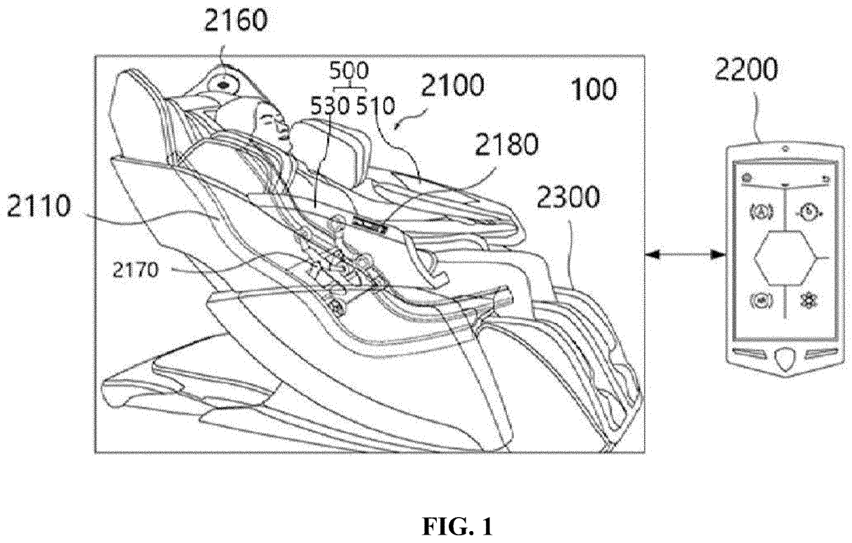

FIG. 1 is a view for depicting a massage apparatus according to an embodiment of the present disclosure.

The massage apparatus 100 according to an embodiment of the present disclosure may include at least one massage unit among a body massage unit 2100 for forming an area for accommodating at least a portion of the body of a user and massaging the body of the user or massaging at least a portion of the body, a leg massage unit 2300 for massaging the user's legs, and an arm massage unit 500 for massaging the user's arms.

For instance, the leg massage unit 2300 may be disposed at a lower portion of the body massage unit 2100 and/or at a front lower portion of the massage apparatus 100. The leg massage unit 2300 includes at least one leg massage unit among a first leg massage unit 2310 disposed at one side of the leg massage unit 2300 to massage one leg of the user and a second leg massage unit 2330 disposed at the other side of the leg massage unit 2300 to massage the other leg of the user.

The arm massage unit 500 may be disposed on at least one among one side and the other side of the body massage unit 2100. For example, the arm massage unit 500 may include at least one among a first arm massage unit 510 disposed at one side (or left side) of the body massage unit 2100 to massage one arm of the user and a second arm massage units 530 disposed at the other side (or right side) of the body massage unit 2100 to massage the other arm of the user. The first and second arm massage units 510 and 530 may operate independently from each other under the control of the control unit 300.

The body massage unit 2100 may provide massage to at least a portion of the body of the user. For example, the body massage unit 2100 may massage at least a portion of the body part among the user's upper body and lower body.

The body massage unit 2100 may include a massage module 2170 for providing a massage function to at least a portion of the body of the user, an audio output module 2160 for providing an audio output of an arbitrary form to the user, a main frame 2110 constituting a framework of the body massage unit 2100, and a user input unit 2180 for receiving any form of input from the user.

The components included in the body massage unit 2100 described above are merely exemplary embodiments, and the body massage unit 2100 may include various configurations besides the above described configuration.

In addition, the shape and structure of the massage apparatus 100 illustrated in FIG. 1 are merely exemplary, and various types of massage apparatuses 100 may also be included within the scope of the present disclosure unless departing from the scope of the present disclosure defined by the claims of the present disclosure.

The body massage unit 2100 may form a space for accommodating the user. The body massage unit 2100 may have a space corresponding to the shape of the body of the user. For example, as illustrated in FIG. 1, the body massage unit 2100 may be implemented as a seating type capable of accommodating the whole body or a portion of the body of the user.

A portion of the body massage unit 2100, which gets in contact with the ground, may include any material or any member (for example, an anti-slip pad or the like) for increasing a frictional force, and may include a wheel for enhancing mobility of the massage apparatus 100.

At least a portion of the body massage unit 2100 may slide. For example, in a case in which the body massage unit 2100 starts massage, at least a portion of the body massage unit 2100 may slide forward. In addition, the body massage unit 2100 may be tilted backward. As a result, the body massage unit 2100 may provide massage while being tilted backward.

According to an embodiment of the present disclosure, the massage apparatus 100 may include at least one air cell (not illustrated). The air cell may be located at a shoulder portion, a pelvis portion, arm massage units, a leg massage unit 2300, and the like, but is not limited thereto, and may be disposed in various portions of the massage apparatus 100.

The massage apparatus 100 may include an air supply unit, and the air supply unit may inflate the air cell by supplying air to the air cell. The air supply unit may be located inside the body massage unit 2100 and may be located in the leg massage unit 2300. Additionally, the air supply unit may be located outside the massage apparatus 100.

The leg massage unit 2300 may include an accommodating space for accommodating the user's leg to massage the user's leg by massaging at least a portion of the leg of the user. For example, the leg massage unit 2300 may include a calf massage unit for massaging the calves of the user and/or a foot massage unit for massaging the user's feet.

The leg massage unit 2300 may adjust the length according to the body characteristics of the user. For example, in a case in which a user who is tall uses the massage apparatus 100, it is necessary to lengthen the leg massage unit 2300 since the user has long calves. Moreover, in a case in which a user who is short uses the massage apparatus 100, it is necessary to shorten the leg massage unit 2300 since the user has short calves.

The massage module 2170 may be provided inside the body massage unit 2100 to provide a mechanical stimulation to the user accommodated in the body massage unit 2100. As illustrated in FIG. 1, the massage module 2170 may move along a main frame 2110 provided inside the body massage unit 2100.

For example, a rack gear may be provided in the main frame 2110 of the body massage unit 2100. The massage module 2170 may provide dynamic stimulation to various parts of the body of the user while moving along the rack gear. The massage module 2170 may include a ball massage unit or a roller massage unit, but is not limited thereto.

The main frame 2110 constitutes a frame of an internal configuration of the body massage unit 2100, and may be formed of a metal material, a plastic material, or the like. For example, the main frame 2110 may be made of iron, an alloy, steel, or the like, but is not limited thereto, and may be made with various rigid materials.

According to an embodiment of the present disclosure, the massage apparatus 100 may include an audio output module 2160. The audio output module 2160 may be provided at various positions. For example, the audio output module 2160 may include a plurality of output units, such as an upper audio output unit disposed on an upper end of a seat portion getting in contact with the user, a front audio output unit attached to a front end of the left and right arm massage units of the seat portion, and/or a rear audio output units attached to rear ends of the arm massage units, but is not limited thereto. In this case, the audio output module 2160 may provide a stereoscopic sound, such as a 5.1 channel, but is not limited thereto.

According to an embodiment of the disclosure, the user may control the massage apparatus 100 by using a massage apparatus control device 2200. The massage apparatus control device 2200 may be connected to the massage apparatus 100 through wired communication and/or wireless communication.

The massage apparatus control device 2200 may include a remote controller, a cellular phone, a personal digital assistant (PDA), and the like, but is not limited thereto, and may include various electronic devices connectable to the massage apparatus 100 through wired or wireless communication.

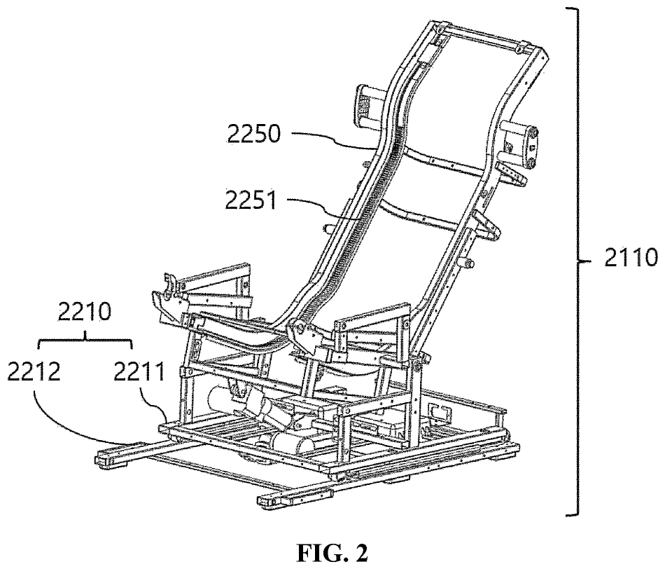

FIG. 2 is a view for depicting a main frame according to an embodiment of the present disclosure.

According to an embodiment of the present disclosure, the main frame 2110 may include an upper frame 2250 on which the massage module 2170 is disposed, and a base frame 2210 supporting the upper frame 2250.

At least a portion of the upper frame 2250 may be provided with a rack gear 2251. The rack gear 2251 is a member for guiding the vertical movement of the massage module 2170, and may include a plurality of valleys and a plurality of crests.

According to an embodiment of the present disclosure, the rack gears 2251 may be disposed at both sides of the upper frame 2250 to face each other, and the massage module 2170 may move along the rack gears 2251.

In FIG. 2, the rack gear 2251 is formed in a vertical direction, but is not limited thereto. The rack gear 2251 may include a rack gear of a back-and-forth direction and a rack gear of a vertical direction. In the present disclosure, the back-and-forth direction refers to a direction heading from the massage module 2170 toward the user or from the user to the massage module 2170, and may also be referred to as a Z-axis direction.

The massage module 2170 may include a gear engaged with the rack gear 2251. More specifically, the massage module 2170 may include gears respectively engaged with the rack gear of the back-and-forth direction and the rack gear of the vertical direction. The gears are rotated by the actuator disposed in the massage module 2170, so that the massage module 2170 may move forward, backward, upward, or downward.

As the massage module 2170 moves forward, the strength of massage may be increased. Moreover, as the massage module 2170 moves backward, the strength of massage may be decreased.

The rack gear 2251 may be formed of a metal material or a plastic material. For example, the rack gear 2251 may be made of iron, steel, alloy, reinforced plastic, melamine resin, phenol resin, or the like, but is not limited thereto.

The upper frame 2250 may be formed in various shapes. For example, the upper frame 2250 may be one selected among an S frame, an L frame, an S&L frame, and a double S&L frame according to the shape of the upper frame, but is not limited thereto.

The S frame refers to a frame in which at least a portion of the upper frame 2250 has a curved shape like “S”. The L frame refers to a frame in which at least a portion of the upper frame 2250 is bent like “L”, the S&L frame refers to a frame having both of the curved shape like “S” and the bent shape like “L”, and the double S&L frame refers to a frame having the curved shape like “L” and the curved shape like “S” formed at two portions.

The base frame 2210 supports the upper frame 2250 of the main frame 2110 and gets in contact with the ground. The base frame 2210 may include a base upper frame 2211 and a base lower frame 2212.

The base upper frame 2211 may support the upper frame 2250, and the base lower frame 2212 may get in contact with the ground. In addition, the base upper frame 2211 may be located in contact with the base lower frame 2212.

According to an embodiment of the present disclosure, the base upper frame 2211 may move along the base lower frame 2212. For example, the base upper frame 2211 may slide forward or backward along the base lower frame 2212. In this case, the upper frame 2250 may be connected to the base upper frame 2211 and move depending on the movement of the base upper frame 2211.

For example, in a case in which the base upper frame 2211 moves forward, the upper frame 2250 may also move forward together. In a case in which the base upper frame 2211 moves backward, the upper frame 2250 may also move backward together. Due to this, the sliding movement of the body massage unit 2100 may be allowed.

Specifically, in order to allow movement of the base upper frame 2211, a moving wheel may be provided beneath the base upper frame 2211. In addition, a guide member capable of guiding the moving wheel may be provided on an upper portion of the base lower frame 2212. The moving wheel provided beneath the base upper frame 2211 may move along the guide member provided on the base lower frame 2212, thereby allowing forward movement or backward movement of the base upper frame 2211.

According to another embodiment of the present disclosure, the massage apparatus 100 may not provide a sliding function, and in this case, the base frame 2210 may not be separated into upper and lower frames.

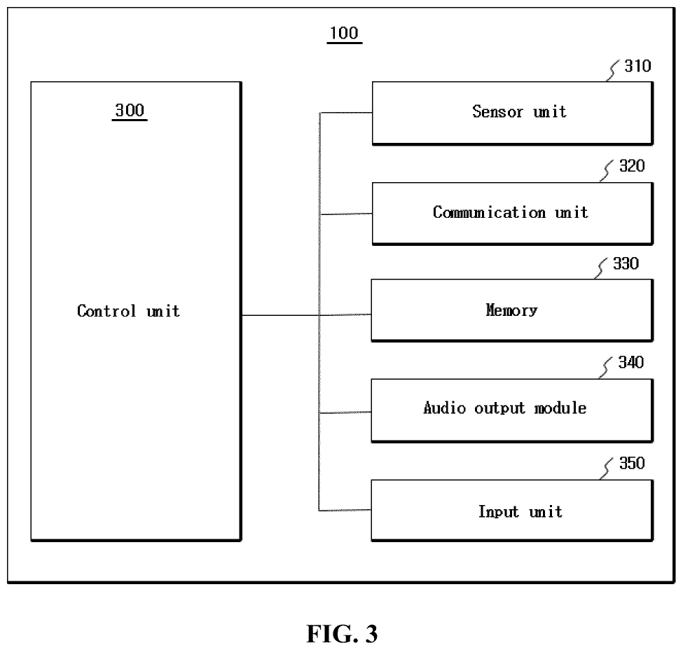

FIG. 3 is a view illustrating a massage apparatus according to an embodiment of the present disclosure.

The massage apparatus 100 may include at least one among a control unit 300, a sensor unit 310, a communication unit 320, a memory 330, an audio output unit 340, and an input unit 2180. The control unit 300 may include at least one processor and a memory. The at least one processor may perform instructions stored in the memory.

The control unit 300 may control the operation of the massage apparatus 100. The control unit 300 may include one processor or may include a plurality of processors. In a case in which the control unit 300 includes a plurality of processors, at least a portion of the plurality of processors may be physically spaced apart from each other. In addition, the massage apparatus 100 is not limited thereto and may be implemented in various ways.

According to an embodiment of the present disclosure, the control unit 300 may control the operation of the massage apparatus 100. For example, the massage apparatus 100 may include a plurality of actuators. The massage apparatus 100 may control the operation of the massage apparatus 100 by controlling the operation of the plurality of actuators. For example, the massage apparatus 100 may include at least one among a driving unit for moving an armrest frame support unit, a massage module moving actuator, at least one actuator included in the massage module, a back angle actuator, a leg angle actuator, a foot massage actuator, a leg length adjusting actuator, and a sliding actuator, and the control unit 300 may control the operation of the massage apparatus 100 by controlling them.

The massage module moving actuator is an actuator that enables vertical movement of the massage module 2170, and the massage module 2170 may move along the rack gear by the operation of the massage module moving actuator.

The back angle actuator is an actuator that adjusts an angle of a portion where the back of the user gets in contact with the massage apparatus 100, and the back angle of the massage apparatus 100 may be adjusted by the operation of the back angle actuator.

The leg angle actuator is an actuator for adjusting an angle of the leg massage unit 2300 of the massage apparatus 100. An angle between the leg massage unit 2300 and the body massage unit 2100 may be adjusted by the operation of the leg angle actuator.

The foot massage actuator is an actuator that operates the foot massage module included in the leg massage unit 2300. The massage apparatus 100 may provide foot massage to the user by using the foot massage actuator.

The massage module 2170 may include at least one actuator, and the control unit 300 may operate at least one actuator to provide various massage motions. For example, the control unit 300 may operate at least one actuator included in the massage module 2170 to provide a tapping massage, a kneading massage, and the like, and may provide various massage operations without being limited thereto.

The leg length adjusting actuator represents an actuator that adjusts the length of the leg massage unit 2300. For example, the control unit 300 may adjust the length of the leg massage unit 2300 to be suitable for the user by using the leg length adjusting actuator, and as a result, the user may receive massage suitable for the user's body shape.

The sliding actuator enables a sliding motion of the massage apparatus 100. For example, a horizontal base upper frame may move forward or backward by the operation of the sliding actuator, and as a result, the upper frame connected to the horizontal base upper frame may also move forward or backward.

The memory 330 may be included in the control unit 300 or may be located outside the control unit 300. The memory 330 may store various pieces of information related to the massage apparatus 100. For example, the memory 330 may include massage control information and personal authentication information, but is not limited thereto.

The memory 330 may be implemented through a non-volatile storage medium capable of continuously storing arbitrary data. For example, the memory 330 may include not only a disk, an optical disk, and a magneto-optical storage device, but also a storage device based on a flash memory and/or a battery-backup memory, but is not limited thereto.

The memory 330 may be a primary storage device, to which a processor directly accesses, like a random access memory (RAM), such as a dynamic random access memory (DRAM), a static random access memory (SRAM), or the like. The memory 330 may mean a volatile storage device in which stored information is instantaneously erased when power is turned off, but is not limited thereto. The memory 330 may be operated by the control unit 300.

The massage apparatus 100 may include a sensor unit 310. The sensor unit 310 may acquire various information using at least one sensor. The sensor unit 310 may be a sensor using a measurement means such as pressure, potential, and optics. For example, the sensor may include a pressure sensor, an infrared sensor, an LED sensor, a touch sensor, and the like, but is not limited thereto.

In addition, the sensor unit 310 may include a biometric information acquisition sensor. The biometric information acquisition sensor may acquire fingerprint information, face information, voice information, iris information, weight information, electrocardiogram information, body composition information, and the like, but is not limited thereto, and may include various pieces of biometric information. For instance, the biometric information acquisition sensor may include at least one among a first bio-signal measuring unit, a second bio-signal measuring unit, a third bio-signal measuring unit, and a fourth bio-signal measuring unit, which will be described later.

According to another embodiment of the present disclosure, the massage apparatus 100 may sense a contact area and/or a contact position with the user through sensors. Furthermore, the massage apparatus 100 may provide a customized massage based on information acquired through sensors. In addition, the massage apparatus 100 may include a communication unit. The communication unit of the massage apparatus 100 may receive a signal from an external device. The control unit 300 may process the received signal to obtain a result signal. The communication unit may output the result signal to the external device.

The communication unit 320 may communicate with a module inside the massage apparatus 100, an external massage apparatus, and/or a user terminal through any type of network. The communication unit may include a wired/wireless access module for network access. The wireless access technology may include, for example, wireless LAN (WLAN) (Wi-Fi), wireless broadband (Wibro), world interoperability for microwave access (Wimax), high speed efficient downlink alliance (HSDPA), and the like. The wired access technology may include, for example, digital subscriber line (XDSL), fibers shop to the home (FTTH), power line communication (PLC), and the like. In addition, the network connection unit may include a short-range communication module to transmit and receive data to and from any device/terminal located at a short distance. For example, the short-range communication technology may include Bluetooth, radio frequency identification (RFID), infrared data security association (IrDA), ultra-wideband (UWB), ZigBee, and the like, but is not limited thereto.

Additionally, the massage apparatus 100 may further include an input unit 350 and/or an output unit. The massage apparatus 100 may receive an input from the user by using the input unit 350. In addition, the massage apparatus 100 may output the processed result of the control unit 300 to the output unit.

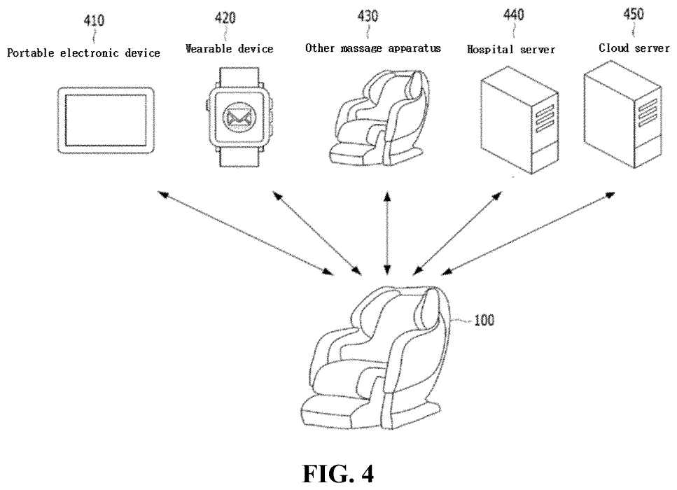

Specifically, the input unit 350 may receive a command related to the operation control of the massage apparatus 100 from the user, and the input unit 350 may be implemented in various forms. For example, the input unit 350 may be provided in the body massage unit 2100, or may be provided in the leg massage unit 2300, but is not limited thereto. Moreover, the input unit 350 may include a user input unit 2180, the massage apparatus control device 2200 of FIG. 1, or various external devices to be described referring to FIG. 4.

The massage apparatus 100 may acquire various commands from the user through the input unit 350. For example, the massage apparatus 100 may receive commands related to section of a massage module, selection of a massage type, selection of massage intensity, selection of massage time, selection of a massage area, selection of a position and an operation of the body massage unit 2100, selection of ON and OFF of power of the massage apparatus 100, selection of whether to operate a thermal function, selection related to sound reproduction, and the like, but is not limited thereto.

The massage apparatus 100 may provide an interface for selecting a massage mode. For example, the input unit 350 or the output unit may include a massage apparatus control device 2200. A medical massage list of various modes related to body improvement may be listed through the massage apparatus control device 2200.

The medical massage mode may include at least one among a concentration mode, a meditation mode, a recovery mode, a stretching mode, a sleep mode, a vital mode, a golf mode, a hip-up mode, an examinee mode, a gravity-free mode, and a growth mode.

According to another embodiment of the present disclosure, the input unit 350 may include buttons of a hot key type and/or selection buttons for selection, cancellation and input, according to preset user setting functions or preset self-setting functions.

The input unit 350 may be implemented as a keypad, a dome switch, a (static pressure type/capacitive) touch pad, a jog wheel, a jog switch, or the like, but is not limited thereto. Furthermore, the input unit 350 may obtain commands through the utterance of the user based on the voice recognition technology.

According to an embodiment of the present disclosure, the output unit may include a display for displaying an operation situation of the massage apparatus 100 or a current state of the user. In this case, the display may include at least one among a liquid crystal display (LCD), a thin film transistor-liquid crystal display (TFT-LCD), an organic light-emitting diode (OLED) display, a flexible display, and a three-dimensional (3D) display, but is not limited thereto.

The output unit may include an audio output unit 340. The audio output unit 340 of FIG. 3 may include the audio output module 2160 of FIG. 1. The audio output unit 340 may provide a user with an audio output of any form. For example, the audio output unit 340 may output a sound source and/or a binaural beat optimized for a massage pattern provided by the massage apparatus 100 to the user, thereby providing a brain stimulation to the user. The audio output unit 340 may output a sound signal received through a network (not illustrated) or stored in an internal/external storage medium (not illustrated). For example, the audio output unit 340 may output a sound source according to the control of the user terminal through a network connection (e.g., Bluetooth connection, etc.) with the user terminal. Additionally, the audio output unit 340 may output a sound signal generated in relation to the operation of the massage apparatus 100.

It will be appreciated by those skilled in the art that the present disclosure may be implemented in combination with other program modules and/or through a combination of hardware and software. For example, the present disclosure may be implemented by a computer-readable medium.

Any of media accessible by a computer may be a computer-readable medium. Such a computer-readable medium may include a volatile medium, a non-volatile medium, a transitory medium, a non-transitory medium, a movable medium, and an unmovable medium. For example, the computer-readable medium may include a computer-readable storage medium and a computer-readable transmission medium.

The computer-readable storage medium includes a volatile medium, a non-volatile medium, a transitory medium, a non-transitory medium, a movable medium, and an unmovable medium implemented by computer-readable commands, a data structure, a program module, or a method or technology for storing information like data. The computer-readable storage medium includes a memory technology, such as a RAM, a ROM, an EEPROM, a flash memory or the like, a magnetic storage device, such as a CD-ROM, a digital video disk (DVD) or the like, a magnetic storage device, such as a magnetic cassette, a magnetic tape, a magnetic disk storage or the like that can be accessed by a computer and that can be used to store desired information, but is not limited thereto.

FIG. 4 is a view illustrating an external device capable of communicating with the massage apparatus 100 according to an embodiment of the present disclosure.

The massage apparatus 100 may communicate with an external device in a wired or wireless manner to transmit and receive various data.

The external device may include a portable electronic device 410, such as an AI speaker, a tablet, or a smartphone. The portable electronic device 410 may be only for the massage apparatus 100 or may be a general-purpose portable electronic device. In addition, the external device may include a wearable device 420 such as a smart watch or a smart band. The external device may not include the massage apparatus 100 currently used by the user but other massage apparatus 430. The external device may include a hospital server 440. The external device may include a personal health record (PHR) server. Furthermore, the external device may include a cloud server 450. The external device may include a medical measurement device, such as an electronic scale, a blood glucose monitoring device, or a blood pressure meter.

Although some examples of the external device are described in the present disclosure, it should be understood that all kinds of devices capable of communicating with the massage apparatus 100 and sending and receiving information with the massage apparatus 100 in a wired or wireless manner may be included in an external device.

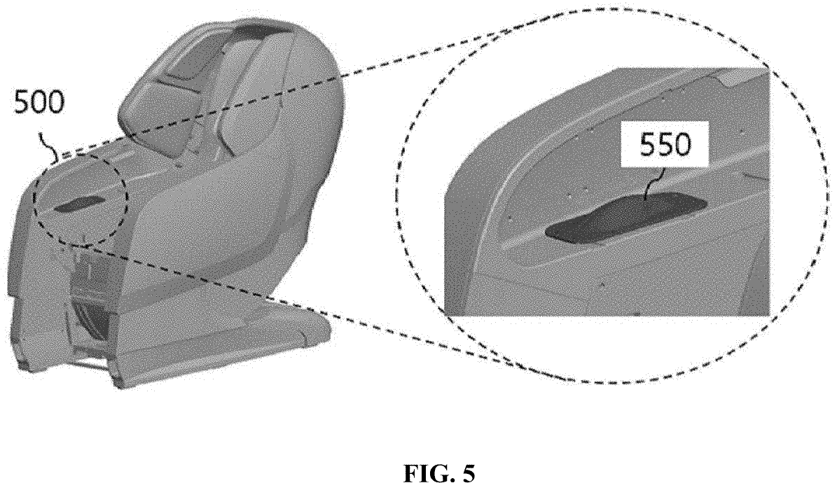

FIG. 5 is a view illustrating a first bio-signal measuring unit according to an embodiment of the present disclosure.

The massage apparatus 100 may include a first bio-signal measuring unit 550. The first bio-signal measuring unit 550 may be disposed (or formed or provided) on the arm massage unit 500. In other words, the arm massage unit 500 may include the first bio-signal measuring unit 550. The first bio-signal measuring unit 550 may be located at an area where the user's palm is placed. Moreover, the first bio-signal measuring unit 550 may include at least one electrode. The user's hand may be naturally positioned on the first bio-signal measuring unit 550 while receiving massage. The first bio-signal measuring unit 550 may measure the user's bio-signal using at least one electrode. The control unit 300 may obtain information on the user's body condition based on the measured bio-signal. The information on the user's body condition acquired by the first bio-signal measuring unit 550 may include body composition and/or electrocardiogram.

Body composition refers to information about body constituent parts. For example, body composition may include information about intracellular water, extracellular water, body water, protein, mineral, body fat, muscle mass, body fat mass, skeletal muscle mass, body fat percentage, BMI, muscle mass by region, body water by region, anasarca, edema by region, body cell mass, bone mineral content, percentage of abdominal fat, cross-sectional area of visceral fat, and basic metabolic rate, but is not limited thereto.

In a case in which the massage apparatus 100 sends a weak alternating current to the human body through the bio-signal measuring unit, the current flows along the highly conductive body water, and the width of a passage through which electricity flows is determined according to the amount of body water. It appears as a measured value called impedance, and the massage apparatus can calculate body composition by using the measured impedance. In this case, the massage apparatus 100 may calculate body composition by using a 2-electrode 4-touch electrode method using two electrodes, and may calculate body composition by using a 4-electrode 8-touch electrode method using four electrodes.

The electrocardiogram may be a record of the electrical activity of the heart. The electrocardiogram may be recorded by electrodes attached to the skin and by devices outside the body. The electrocardiogram may be recorded with an ammeter by inducing an action current generated from the myocardia according to the heartbeat to two appropriate electrodes on the body surface. The electrocardiogram is used not only to measure the rate and regularity of heartbeats, but also to determine the size and location of the heart and whether there is any damage to the heart.

The first bio-signal measuring units 550 may be respectively disposed on the first and second arm massage units 510 and 530. The first bio-signal measuring unit disposed on each arm massage unit may include two electrodes. Accordingly, the first bio-signal measuring unit 550 may have a total of four electrodes including the left and right sides. The control unit 300 may measure a bio-signal of the user by using at least two electrodes among the electrodes included in the first bio-signal measuring unit located on the user's hand, the second bio-signal measuring unit located on the user's foot, and/or the fourth bio-signal measuring unit.

The first bio-signal measuring unit 550 can be moved back and forth based on the location of the user's hand. For example, the control unit 300 may receive a signal from a sensor for measuring the position of the user's hand. In order to measure the user's hand, at least one among an imaging device (or a camera), a pressure sensor, an optical sensor (e.g., an infrared sensor, etc.), an ultrasonic sensor, and an electrode may be used, but is not limited thereto, and various sensors capable of recognizing the position of the user's hand may be used. The control unit 300 may automatically move the first bio-signal measuring unit 550 based on the position of the user's hand. The control unit 300 may move the first bio-signal measuring unit 550 forward and backward. Here, the terms of forward and backward mean the forward direction and the backward direction of the user (or the forward direction and the backward direction of the massage apparatus 100) when the user sits on the massage apparatus 100. The user may comfortably put the hand on the first bio-signal measuring unit 550.

However, the present disclosure is not limited to the first bio-signal measuring unit 550 moving automatically. The user may manually move the first bio-signal measuring unit 550. The user may pull or push the first bio-signal measuring unit 550 to move it. The first bio-signal measuring unit 550 may move forward and backward by the user's force.

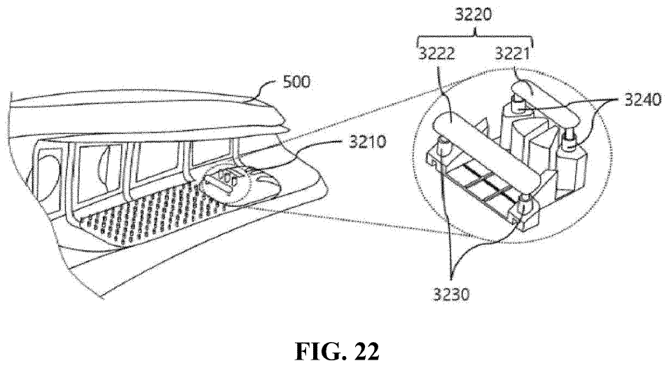

FIG. 22 is a view for depicting a third bio-signal measuring unit according to an embodiment of the present disclosure.

The massage apparatus 100 may include a third bio-signal measuring unit 3210. FIG. 22 illustrates the third bio-signal measuring unit 3210 disposed inside the massage apparatus 100. The third bio-signal measuring unit 3210 may replace the first bio-signal measuring unit 550. In other words, the arm massage unit 500 may include at least one among the first bio-signal measuring unit 550 and the third bio-signal measuring unit 3210. For example, the first arm massage unit 510 may include any one among the first bio-signal measuring unit 550 and the third bio-signal measuring unit 3210, and the second arm massage unit 530 may include a bio-signal measuring unit which is different from or identical for the bio-signal measuring unit included in the first arm massage unit 510, among the first bio-signal measuring unit 550 and the third bio-signal measuring unit 3210.

The third bio-signal measuring unit 3210 may be at least partially hidden by a cover sheet. However, FIG. 22 illustrates a state in which the cover sheet is removed for convenience of description. The cover sheet may be a type of housing that covers a portion of the arm massage unit 500 so that the arm massage unit 500 looks clean. The cover sheet may be made of polyurethane (PU), polystyrene, or a sponge material.

The third bio-signal measuring unit 3210 is disposed (or may be formed or prepared) on the arm massage unit 500. The third bio-signal measuring unit 3210 may be located at an area where the user's palm is placed. Furthermore, the third bio-signal measuring unit 3210 may include at least one electrode. The user's palm may be naturally positioned on the third bio-signal measuring unit 3210 while the user gets massage. The third bio-signal measuring unit 3210 may measure the user's bio-signal by using at least one electrode. The control unit 300 may obtain information on the user's body condition based on the measured bio-signal. The information on the user's body condition acquired by the third bio-signal measuring unit 3210 may include body composition and/or electrocardiogram.

The massage apparatus 100 may include two third bio-signal measuring units 3210. The third bio-signal measuring units 3210 may be respectively disposed on the first and second arm massage units 510 and 530. The third bio-signal measuring unit disposed on each arm massage unit may include two electrodes. Accordingly, the third bio-signal measuring units 3210 may have a total of four electrodes including the left and right sides. The control unit 300 may measure the user's bio-signal by using at least two among the electrodes included in the third bio-signal measuring unit 3210 located on the user's hand, the second bio-signal measuring unit 610 located on the user's foot, and/or the fourth bio-signal measuring unit 3310.

The third bio-signal measuring unit 3210 may be movable forward and backward like the first bio-signal measuring unit 550 or may not be movable forward and backward like the first bio-signal measuring unit 550. The massage apparatus 100 may include any one among the first bio-signal measuring unit 550 and the third bio-signal measuring unit 3210. Since the first and third bio-signal measuring units 550 and 3210 are located where the user can comfortably place the hand, the user can comfortably select any one of the first and third bio-signal measuring units 550 and 3210 to put the hand thereon.

Hereinafter, for convenience of description, it is assumed that the massage apparatus 100 includes the first bio-signal measuring unit 550. All of the contents of the first bio-signal measuring unit 550 described below may be applied to the third bio-signal measuring unit 3210.

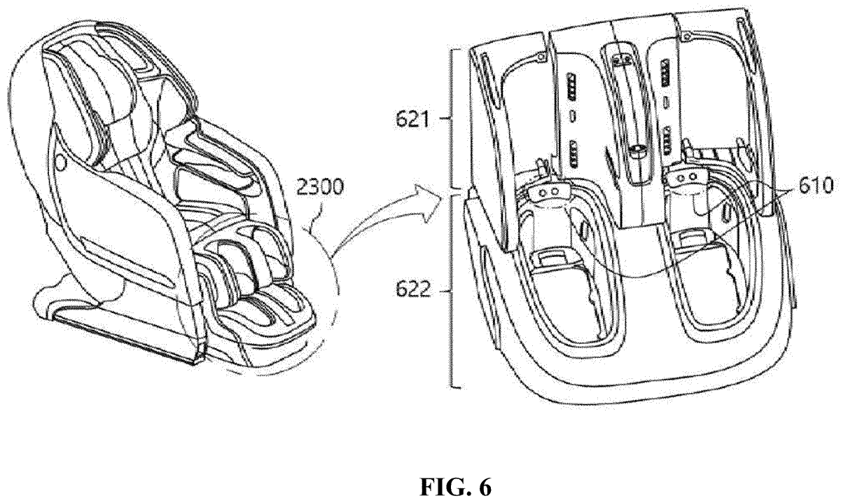

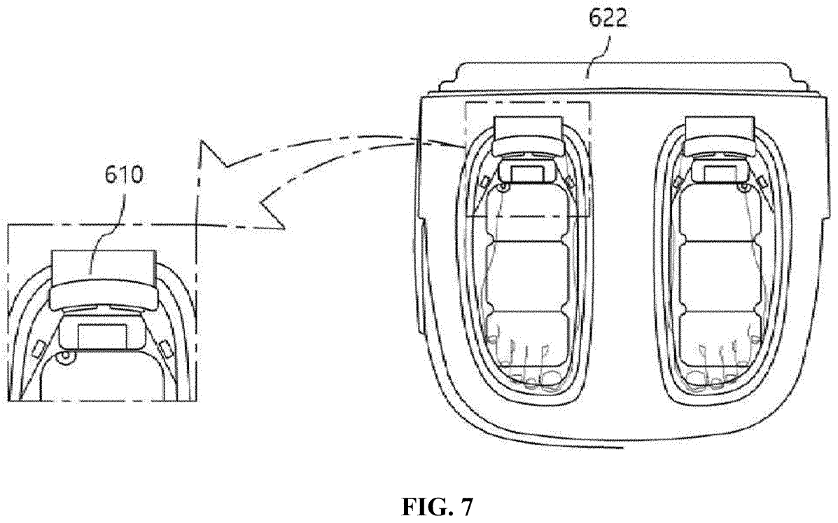

FIG. 6 is a view for depicting a second bio-signal measuring unit according to an embodiment of the present disclosure. Moreover, FIG. 7 is a view for depicting a method for a user to measure body composition through a second bio-signal measuring unit according to an embodiment of the present disclosure.

The massage apparatus 100 may include a second bio-signal measuring unit 610. The second bio-signal measuring unit 610 may be arranged (or formed, disposed, or provided) on the foot massage unit 622. In other words, the leg massage unit 2300 may include the second bio-signal measuring unit 550. As described above, the foot massage unit 622 may be included in the leg massage unit 2300. The second bio-signal measuring unit 610 is located on the back of the user's ankle and may include at least one electrode.

The massage apparatus 100 may measure body composition using a bipolar electrode method by utilizing the first bio-signal measuring unit 550 or the second bio-signal measuring unit 610. The massage apparatus 100 may measure body composition using a quadrupolar electrode method by utilizing both of the first bio-signal measuring unit 550 and the second bio-signal measuring unit 610, but is not limited thereto, and may measure body composition using a multi-polar electrode method.

In addition, according to another embodiment of the present disclosure, the massage apparatus 100 may measure body composition using the bipolar electrode method by individually utilizing the first bio-signal measuring unit 550 and the second bio-signal measuring unit 610, and may obtain the user's body composition by selecting a highly reliable measurement result through a reliability test.

Furthermore, the massage apparatus 100 may measure an electrocardiogram by utilizing the first bio-signal measuring unit 550 and/or the second bio-signal measuring unit 610.

The second bio-signal measuring unit 610 may be arranged on at least a portion of the leg massage unit 2300 so as to get in contact with at least a portion of the leg of the user when the user sits on the massage apparatus 100.

For instance, the leg massage unit 2300 is divided into a calf massage unit 621 massaging the user's calves and a foot massage unit 622 massaging the user's feet, and the second bio-signal measuring unit 610 may be located between the calf massage unit 621 and the foot massage unit 622. In addition, the second bio-signal measuring unit 610 may be provided on at least a portion of the foot massage unit 622, or may be located on at least a portion of the calf massage unit 621.

The second bio-signal measuring unit 610 may come into contact with a portion between the user's calf and foot in a case in which the user sits on the massage apparatus 100. For example, in a case in which the user sits on the massage apparatus 100, the second bio-signal measuring unit 610 may come into contact with the back of the user's ankle.

The second bio-signal measuring units 610 may be respectively located on the left and right leg massage units 2300. The second bio-signal measuring unit of the left side may include two electrodes. Additionally, the second bio-signal measuring unit of the right side may include two electrodes. The second bio-signal measuring unit may have a total of four electrodes including the left and right sides. The control unit 300 may measure the user's bio-signal by using at least one among the electrodes included in the first bio-signal measuring unit 550 or the third bio-signal measuring unit 3210 located on the user's hand and at least one among the electrodes included in the second bio-signal measuring unit 610 located on the user's foot.

The second bio-signal measuring unit 610 is movable forward and backward. More specifically, at least one electrode included in the second bio-signal measuring unit 610 is movable forward and backward. The user may move the second bio-signal measuring unit 610. The second bio-signal measuring unit 610 may be usually located in front by a spring. However, in a case in which the user sits on the massage apparatus 100 and puts the legs or feet on the second bio-signal measuring unit 610, the second bio-signal measuring unit 610 may be moved backward by the user's legs or feet. Here, the terms of ‘front’ and ‘back’ may mean the front and the back of the foot of the user. For instance, the ‘front’ may mean the user's toes, and the ‘back’ may mean the user's heel. In addition, while the user is sitting on the massage apparatus 100, the front and back may mean the front and back of the user. That is, the front may mean a toe direction (or the forward direction of the leg massage unit 2300) and the back may mean a heel direction (or the backward direction of the leg massage unit 2300). Additionally, in a state in which the user lies down with his/her legs stretched on the massage apparatus 100, the front may mean the sky direction, and the back may mean the ground direction. As described above, since the second bio-signal measuring unit 610 or the electrode may move forward and backward, at least one electrode may get in close contact with the user's foot or leg to measure a bio-signal.

The second bio-signal measuring unit 610 may be used in a case in which an angle 3351 that the leg massage unit 2300 is perpendicular to the ground surface is greater than or equal to a predetermined angle. The massage apparatus 100 may incline the leg massage unit 2300. More specifically, the basic state of the massage apparatus 100 may be a state in which the leg massage unit 2300 is substantially perpendicular to the ground. The massage apparatus 100 may tilt the leg massage unit 2300 to make the user lie down. As the leg massage unit 2300 is inclined, the user's knees and legs may be stretched. In addition, as the leg massage unit 2300 is inclined, the angle between the vertical line of the ground and the leg massage unit 2300 may gradually increase. In a case in which the angle between the vertical line of the ground and the leg massage unit 2300 is greater than or equal to a predetermined angle, the massage apparatus 100 may measure the bio-signal by using the second bio-signal measuring unit 610. The user's legs may be forced down by gravity. Therefore, in a case in which the angle between the vertical line of the ground and the leg massage unit 2300 is greater than or equal to the predetermined angle, the user's heel gets in close contact with the second bio-signal measuring unit 610, and the massage apparatus 100 may accurately measure the user's bio-signal. Additionally, in a case in which the angle between the vertical line of the ground and the leg massage unit 2300 is less than the predetermined angle, the massage apparatus 100 may measure the bio-signal by using the fourth bio-signal measuring unit 3310. The fourth bio-signal measuring unit 3310 will be described in more detail later.

The control unit 300 may obtain information on the user's body condition based on the bio-signal acquired from at least two bio-signal measuring units among the first bio-signal measuring units 550 to the fourth bio-signal measuring unit 3310. The bio-signal may be a value measured by applying current or voltage to the user's body. For example, the bio-signal may include at least one among an impedance, a current value, or a voltage value obtained from at least two bio-signal measuring units among the first bio-signal measuring units 550 to the fourth bio-signal measuring unit 3310. The control unit 300 may obtain information on the biological state based on the bio-signal. Information on the body condition may include body composition and/or electrocardiogram.

Biometric information for obtaining body composition and/or electrocardiogram included in information on the body condition may be duplicated. The control unit 300 may acquire both body composition information and electrocardiogram information by measuring biometric information once without separately measuring biometric information to obtain body composition information and electrocardiogram information, but is not limited thereto. The control unit 300 may separately measure a bio-signal for acquiring body composition information and a bio-signal for acquiring electrocardiogram information.

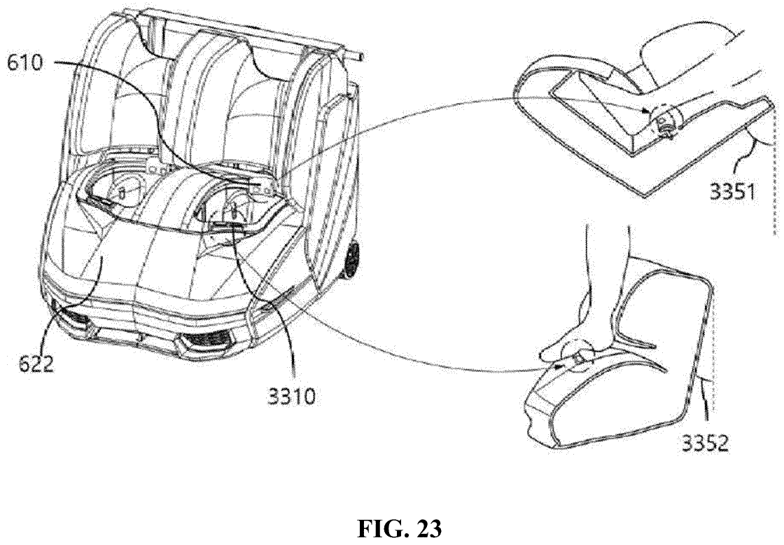

FIG. 23 is a view for depicting a second bio-signal measuring unit and a fourth bio-signal measuring unit according to an embodiment of the present disclosure.

As illustrated in FIGS. 6 and 7, the massage apparatus 100 may include the second bio-signal measuring unit 610, but is not limited thereto. Referring to FIG. 23, the massage apparatus 100 may include a fourth bio-signal measuring unit 3310 instead of the second bio-signal measuring unit 610. Moreover, the massage apparatus 100 may include both of the second bio-signal measuring unit 610 and the fourth bio-signal measuring unit 3310. In other words, the leg massage unit 2300 may include at least one among the second bio-signal measuring unit 610 and the fourth bio-signal measuring unit 3310. For instance, the first and second leg massage units 2310 may include at least one among the second bio-signal measuring unit 610 and the fourth bio-signal measuring unit 3310. In this instance, the bio-signal measuring units included in the first and second leg massage units 2310 may be identical for each or different from each.

The fourth bio-signal measuring unit 3310 may be formed on the foot massage unit 622. As described above, the foot massage unit 622 may be included in the leg massage unit 2300. The fourth bio-signal measuring unit 3310 may be formed on the upper surface of a housing of the foot massage unit 622. Additionally, the fourth bio-signal measuring unit 3310 may include at least one electrode. For instance, the fourth bio-signal measuring unit 3310 may include at least one electrode for measuring a fourth bio-signal.

The massage apparatus 100 may measure body composition using a bipolar electrode method or a quadrupolar electrode method by utilizing at least two among the first bio-signal measuring unit 550, the second bio-signal measuring unit 610, the third bio-signal measuring unit 3210, and the fourth bio-signal measuring unit 3310, but is not limited thereto. The massage apparatus 100 may measure body composition using a multipolar electrode method.

In addition, according to another embodiment of the present disclosure, the massage apparatus 100 may measure body composition using the bipolar electrode by selecting two among the first bio-signal measuring unit 550, the second bio-signal measuring unit 610, the third bio-signal measuring unit 3210, and the fourth bio-signal measuring unit 3310, and may obtain the user's body composition by selecting a highly reliable measurement result through a reliability test. The massage apparatus 100 may obtain body composition values by the at least two bio-signal measuring units selected among the first bio-signal measuring unit 550, the second bio-signal measuring unit 610, the third bio-signal measuring unit 3210, and the fourth bio-signal measuring unit 3310. More specifically, the massage apparatus 100 may obtain body composition values by selecting at least two bio-signal measuring units selected among the first bio-signal measuring unit 550, the second bio-signal measuring unit 610, the third bio-signal measuring unit 3210, and the fourth bio-signal measuring unit 3310. For instance, in a case in which the massage apparatus 100 obtains the body composition values by selecting two bio-signal measuring units, the combination of the bio-signal measuring units may the first bio-signal measuring unit 550 and the second bio-signal measuring unit 610, the first bio-signal measuring unit 550 and the third bio-signal measuring unit 3210, the first bio-signal measuring unit 550 and the fourth bio-signal measuring unit 3310, the second bio-signal measuring unit 610 and the third bio-signal measuring unit 3210, the second bio-signal measuring unit 610 and the fourth bio-signal measuring unit 3310, and the third bio-signal measuring unit 3210 and the fourth bio-signal measuring unit 3310. The massage apparatus 100 may obtain the body composition values with respect to each combination of the bio-signal measuring units.

The fourth bio-signal measuring unit 3310 may be used in a case in which an angle 3352 that the leg massage unit 2300 is perpendicular to the ground surface is less than a predetermined angle. The massage apparatus 100 may incline the leg massage unit 2300. More specifically, the basic state of the massage apparatus 100 may be a state in which the leg massage unit 2300 is substantially perpendicular to the ground. In this instance, the user's knees are in a bent state, and the user's fibulas are substantially perpendicular to the ground. The massage apparatus 100 may tilt the leg massage unit 2300 to make the user lie down. As the leg massage unit 2300 is inclined, the user's knees and legs may be stretched. In addition, as the leg massage unit 2300 is inclined, the angle between the vertical line of the ground and the leg massage unit 2300 may gradually increase. In a case in which the angle between the vertical line of the ground and the leg massage unit 2300 is greater than or equal to a predetermined angle, the massage apparatus 100 may measure the bio-signal by using the second bio-signal measuring unit 610. In a case in which the angle between the vertical line of the ground and the leg massage unit 2300 is less than the predetermined angle, the massage apparatus 100 may measure the bio-signal by using the fourth bio-signal measuring unit 3310. In the case in which the angle between the vertical line of the ground and the leg massage unit 2300 is less than the predetermined angle, the user's calves may be substantially perpendicular to the ground. In addition, since the user's legs are forced by gravity, the sole surface of the user may come into close contact with the fourth bio-signal measuring unit 3310, and the massage apparatus 100 may accurately measure the user's bio-signal.

The massage apparatus 100 may output a usable measuring unit among the second bio-signal measuring unit 610 and the fourth bio-signal measuring unit 3310 into a sound or an image according to the angle between the vertical line of the ground and the leg massage unit 2300. The user can measure the bio-signal by using any one among the second bio-signal measuring unit 610 and the fourth bio-signal measuring unit 3310 according to a guide output from the massage apparatus 100.

The massage apparatus 100 may obtain an average value of the body composition values with respect to the combination of the bio-signal measuring units. Moreover, the massage apparatus 100 may determine a value most similar to the average value of the body composition values with respect to the combination as a highly reliable measurement result. The similar value may be one among the body composition values by the two bio-signal measuring unit selected among the first bio-signal measuring unit 550, the second bio-signal measuring unit 610, the third bio-signal measuring unit 3210, and the fourth bio-signal measuring unit 3310, but is not limited thereto. The massage apparatus 100 may determine the average value of the body composition values with respect to the combination as a highly reliable measurement result.

In addition, the massage apparatus 100 may measure an electrocardiogram by utilizing at least one among the first bio-signal measuring unit 550, the second bio-signal measuring unit 610, the third bio-signal measuring unit 3210, and the fourth bio-signal measuring unit 3310.

The fourth bio-signal measuring unit 3310 is disposed on at least a portion of the leg massage unit 2300. In a case in which the user sits on the massage apparatus 100 and puts the legs on the fourth bio-signal measuring unit 3310, the user's sole may come into contact with the electrode of the fourth bio-signal measuring unit 3310.

The massage apparatus may include two fourth bio-signal measuring units 3310. The fourth bio-signal measuring units 3310 may be respectively located on the left leg massage unit 2300 and the right leg massage unit 2300. The fourth bio-signal measuring unit of the left side may include two electrodes. Furthermore, the fourth bio-signal measuring unit of the right side may include two electrodes. The fourth bio-signal measuring unit may have a total of four electrodes including the left and right sides. The control unit 300 may measure the user's bio-signal by using at least two among the electrodes included in the first bio-signal measuring unit 550, the second bio-signal measuring unit 610, the third bio-signal measuring unit 3210, and the fourth bio-signal measuring unit 3310.

The control unit 300 may obtain information the user's body condition based on the bio-signal obtained from at least one among the first bio-signal measuring unit 550, the second bio-signal measuring unit 610, the third bio-signal measuring unit 3210, and the fourth bio-signal measuring unit 3310. For example, the control unit 300 may measure the user's bio-signal by using at least two among total four third bio-signal measuring electrodes 3220 and at least two among total four fourth bio-signal measuring electrodes, and obtains information on the user's body condition based on the user's bio-signal.

The bio-signal may be a value measured by applying current or voltage to the user's body. For example, the bio-signal may include at least one among an impedance, a current value and a voltage value obtained from at least one among the first bio-signal measuring unit 550, the second bio-signal measuring unit 610, the third bio-signal measuring unit 3210, and the fourth bio-signal measuring unit 3310. The control unit 300 may obtain information on the body condition based on the bio-signal. Information on the body condition may include body composition and/or electrocardiogram.

Biometric information for obtaining body composition and/or electrocardiogram included in information on the body condition may be duplicated. The control unit 300 may acquire both body composition information and electrocardiogram information by measuring biometric information once without separately measuring biometric information to obtain body composition information and electrocardiogram information, but is not limited thereto. The control unit 300 may separately measure a bio-signal for acquiring body composition information and a bio-signal for acquiring electrocardiogram information.

Hereinafter, components included in the first bio-signal measuring unit 550 and the second bio-signal measuring unit 610 will be described in detail.

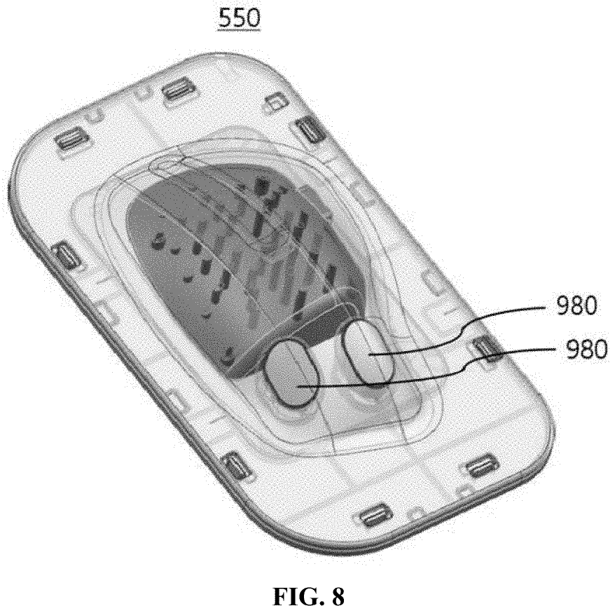

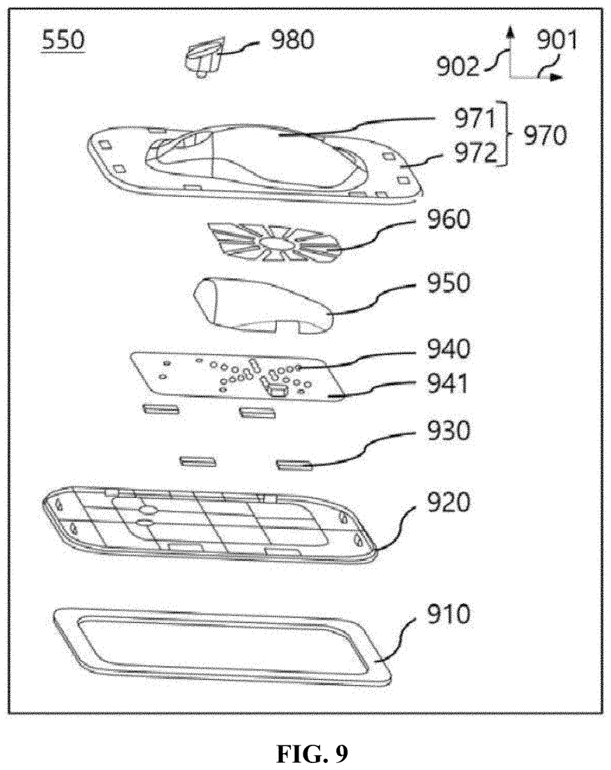

FIG. 8 is a perspective view of a first bio-signal measuring unit according to an embodiment of the present disclosure. FIG. 9 is a view for depicting components included in the first bio-signal measuring unit according to an embodiment of the present disclosure.

In FIG. 9, the front 901 may mean the front side (or the forward direction of the massage apparatus 100) when the user sits on the massage apparatus 100. In addition, the upper side 902 may mean the upper side (or the upward direction of the massage apparatus 100) when the user sits on the massage apparatus 100.

The first bio-signal measuring unit 550 may include a plate 910. The plate 910 may be fixed on the housing of the arm massage unit 500. For example, the plate 910 may be fixed on at least one housing among the first and second arm massage units 510 and 530. However, the third bio-signal measuring unit 3210 may be arranged on at least one among the housing of the first arm massage unit 510 and the housing of the second arm massage unit 530. For instance, an air cell is arranged on at least one among the housing of the first arm massage unit 510 and the housing of the second arm massage unit 530, and the third bio-signal measuring unit 3210 may be arranged on the air cell.

The plate 910 may be made of a metal material. The plate may have a shape elongated to the front 901 or to the back (in the backward direction of the massage apparatus 100). The plate 910 may have a doughnut shape. That is, the plate 910 may have a hole.

The first bio-signal measuring unit 550 may include a lower cover 920. The lower cover 920 may be put on the plate. As described above, the plate 910 may be fixed to the housing of the arm massage unit. However, the lower cover 920 may not be fixed to the plate 910. The lower cover 920 may be slidable on the plate 910.

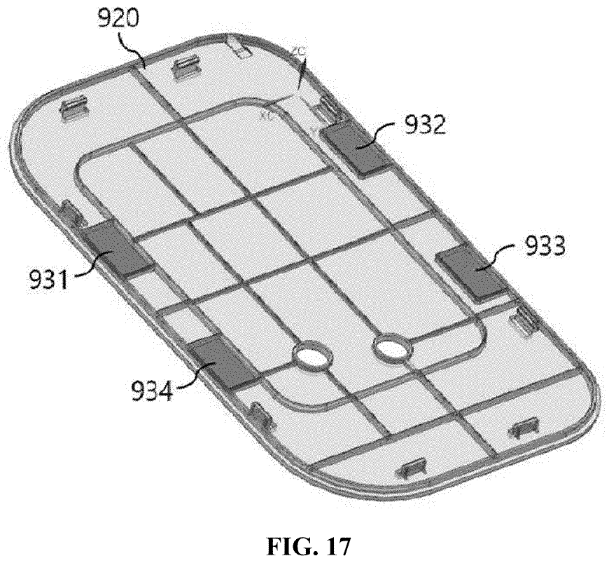

The first bio-signal measuring unit 550 may include at least two magnets 930. The at least two magnets 930 may be fixed on the lower cover 920. Referring to FIG. 17, the location of the magnets 930 according to an embodiment of the present disclosure will be described.

FIG. 17 is a view for depicting magnets according to an embodiment of the present disclosure.

Referring to FIG. 17, The at least two magnets 930 may be disposed at both sides of the lower cover 920. The at least two magnets 930 may be fixed on the lower cover 920. The at least two magnets 930 may include a first magnet 931, a second magnet 932, a third magnet 933, and a fourth magnet 934. The first magnet 931 and the fourth magnet 934 may be located at the left edge of the lower cover 920. The second magnet 932 and the third magnet 933 may be located at the right edge of the lower cover 920.

Referring to FIG. 9, the at least two magnets 930 may face the plate 910. The at least two magnets 930 may not touch the plate 910 by the lower cover 920. However, the at least two magnets 930 and the plate 910 may attract each other by a magnetic force. One of the at least two magnets 930 may be located at the left edge of the lower cover 920, and the other one may be located at the right edge of the lower cover 920.

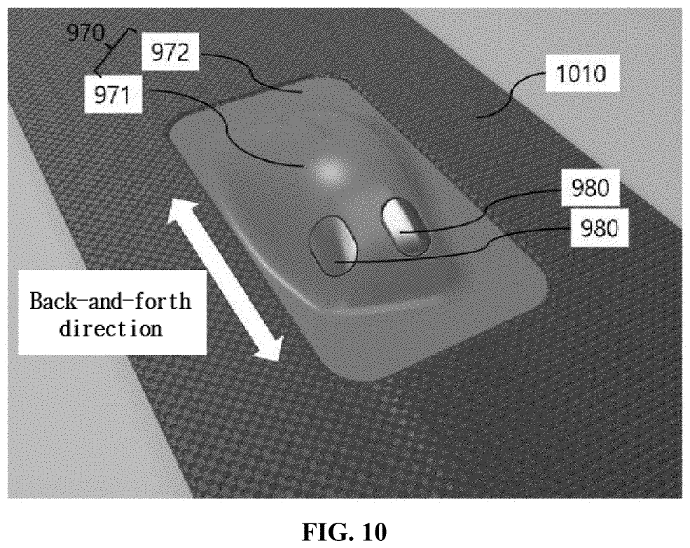

Due to the at least two magnets 930, the lower cover 920 may move forward 901 or backward on the plate 910. The movement of the first bio-signal measuring unit 550 will be described with reference to FIG. 10. Referring to FIG. 9, the first bio-signal measuring unit 550 may include an upper cover 970. The upper cover 970 may cover the lower cover 920 to protect various components on the lower cover 920. The upper cover 970 may be coupled with the lower cover 920. The upper cover 970 may include a palm support 971. The palm support 971 may have an upward convex shape so that the user can put comfortably put the hand on the palm support.

The first bio-signal measuring unit 550 may include a first bio-signal measuring electrode 980. The first bio-signal measuring electrode 980 may have very low resistance (or impedance). Additionally, the first bio-signal measuring electrode 980 may be plated with nickel or chrome. Furthermore, the first bio-signal measuring electrode 980 may be a silicon electrode. The silicon electrode may be an electrode using silicon and silver nanowire. The silver nanowire has excellent conductivity and the flexible nature. Since the silicon electrode is flexible, even if it touches the user's skin, the user does not feel cold and hard, but may feel comfortable. In addition, the silicon electrode is easy to have a desired shape.

The first bio-signal measuring electrode 980 may touch a portion of the palm of the user. The first bio-signal measuring electrode 980 may be inserted into a hole formed in the upper cover 970.

Two first bio-signal measuring electrodes 980 may be included in the first bio-signal measuring unit 550 of the left side. Moreover, two first bio-signal measuring electrodes 980 may be included in the first bio-signal measuring unit 550 of the right side. Therefore, the bio-signal measuring units 550 of the left side and the right side may have a total of four first bio-signal measuring electrodes 980.

The control unit 300 may measure the user's bio-signal by using at least one among the first bio-signal measuring electrode 980 and the second bio-signal measuring electrode 610. In addition, the control unit 300 may obtain information on the body condition based on the bio-signal. The first bio-signal measuring electrode 980 may touch the user. The first bio-signal measuring electrode 980 may be electrically connected to the control unit 300. The control unit 300 may obtain the bio-signal based on the signal from the bio-signal measuring electrode 980. Additionally, the control unit 300 may obtain information on the user's body condition based on the bio-signal.

Referring to FIG. 22, the third bio-signal measuring unit 3210 may include the third bio-signal measuring electrode 3220.

The third bio-signal measuring electrode 3320 may be very lower in resistance. Furthermore, the third bio-signal measuring electrode 3320 may be plated with nickel or chrome. Furthermore, the third bio-signal measuring electrode 3320 may be a silicon electrode. The silicon electrode may be an electrode using silicon and silver nanowire. The silver nanowire has excellent conductivity and the flexible nature. Since the silicon electrode is flexible, even if it touches the user's skin, the user does not feel cold and hard, but may feel comfortable. In addition, the silicon electrode is easy to have a desired shape.

The third bio-signal measuring electrode 3320 may include a third-first bio-signal measuring electrode 3221 and a third-second bio-signal measuring electrode 3222. The third-first bio-signal measuring electrode 3221 and the third-second bio-signal measuring electrode 3222 may be elongated from the left to the right. Here, the left and the right may mean the left and the right when the user sits on the massage apparatus.

The third-first bio-signal measuring electrode 3221 may face upward to touch the user's hand. Furthermore, the third-first bio-signal measuring electrode 3221 may be fixed to the housing of the arm massage unit. In addition, the third-second bio-signal measuring electrode 3222 may be longer than the third-first bio-signal measuring electrode. The third-second bio-signal measuring electrode 3222 may be located at the back of the third-first bio-signal measuring electrode 3221. Here, the back may mean the back when the user sits on the massage apparatus 100. The third-second bio-signal measuring electrode 3222 may face upward to get in touch with the user's hand, and may be fixed to the housing of the arm massage unit.

Bottom surfaces of the third-first bio-signal measuring electrode 3221 and the third-second bio-signal measuring electrode 3222 may be coupled with two electrode supports extending upward and downward. Here, ‘downward’ may mean a direction to face the ground, and ‘upward’ may mean the opposite direction of the downward direction. The bottom surface of the third-first bio-signal measuring electrode 3221 may be coupled with two supports 3240. Furthermore, the bottom surface of the third-second bio-signal measuring electrode 3222 may be coupled with two supports 3230. The electrode supports 3230 and 3240 may be screw-coupled with the housing of the arm massage unit. Therefore, the third bio-signal measuring electrode 3220 may be fixed to the arm massage unit.

The third bio-signal measuring unit 3210 may also include an upper cover 970. The upper cover 970 may cover the lower cover 920 to protect various components and structures on the lower cover 920. The upper cover 970 may be coupled with the lower cover 920. The upper cover 970 may include a palm support 971. The palm support 971 may have an upward convex shape so that the user can put comfortably put the hand on the palm support. The lower cover 920 of the third bio-signal measuring unit 3210 may be fixed to the arm massage unit. Therefore, the third bio-signal measuring unit 3210 may not move forward and backward. The massage apparatus 100 may include only one among the first bio-signal measuring unit 550 and the third bio-signal measuring unit 3210.

The electrode supports 3230 and 3240 may be surrounded by filler. The upper surfaces of the third-first bio-signal measuring electrode 3221 and the third-second bio-signal measuring electrode 3222 may protrude from the filler. Therefore, the third bio-signal measuring electrode 3220 may touch a portion of the palm of the user. The filler may be made of polyurethane (PU), polystyrene, or a sponge material. The upper cover 970 may be located on the filler. The third bio-signal measuring electrode 3220 may be arranged through the hole of the upper cover 970, and the user's palm may touch the third bio-signal measuring electrode 3220.

The third bio-signal measuring unit of the left side may include two third bio-signal measuring electrodes. Moreover, the third bio-signal measuring unit of the right side may include two third bio-signal measuring electrodes. Therefore, the third bio-signal measuring units 3210 of the left side and the right side may include a total of four third bio-signal measuring electrodes 3220.

The control unit 300 may measure the user's bio-signal by using at least one among electrodes of the third bio-signal measuring electrode 3220, the second bio-signal measuring unit 610, and the fourth bio-signal measuring electrode 3410. Additionally, the control unit 300 may acquire information on the body condition based on the bio-signal. The third bio-signal measuring electrode 3220 may get in contact with the user. The third bio-signal measuring electrode 3220 may be electrically connected with the control unit 300. The control unit 300 may acquire a bio-signal based on a signal from the third bio-signal measuring electrode 3220. In addition, the control unit 300 may acquire information on the user's body condition based on the bio-signal.

Referring to FIG. 9, the first bio-signal measuring unit 550 or the third bio-signal measuring unit 3210 may include a plurality of LEDs 940 for treatment. The plurality of LEDs 940 for treatment may be fixed on the lower cover 920. The plurality of LEDs 940 for treatment may emit light based on a control signal of the control unit 300. The plurality of LEDs 940 for treatment may be located on an LED control PCB 941. The LED control PCB 941 may include a processor and a memory. The control unit 300 may send the control signal to the LED control PCB 941. The LED control PCB 941 may turn on or off at least a portion of the LEDs 940 for treatment based on the control signal.

The plurality of LEDs 940 for treatment may emit light of a wavelength good for articulation health of the user. For instance, the wavelength of light emitted from the plurality of LEDs 940 for treatment may be in a range of 0.5 um (micrometer) to 2.5 um. More specifically, the wavelength of light emitted from the plurality of LEDs 940 for treatment may be in a range of 800 nm (nanometer) to 1000 nm. In addition, the power density of light emitted from the LEDs 940 may be in a range of 2.0 to 3.0 mW/cm², and the energy density may be in a range of 11.00 to 12.00 J/cm².

Since the user's hand is positioned on the first bio-signal measuring unit 550 or the third bio-signal measuring unit 3210, the plurality of LEDs 940 for treatment may emit light toward the user's palm. The joints of the hand of the user may be recovered by the light of the plurality of LEDs 940 for treatment. More specifically, the light of the plurality of LEDs 940 for treatment may be directed toward the user's fingers. The user may apply light of the LEDs 940 to the fingers to promote joint health.

The plurality of LEDs 940 for treatment may be located below the upper cover 970. At least a portion of the upper cover 970 may be transparent so that the light of the plurality of LEDs 940 for treatment is transmitted to the user's hand. Alternatively, at least a portion of the upper cover 970 may be translucent. Alternatively, at least a portion of the upper cover 970 may include a hole so that the light of the LEDs 940 is transmitted to the user's hand. The upper cover 970 may be made of a material that transmits only specific wavelengths. For example, the upper cover 970 may be made of a material that transmits only the wavelength of light emitted from the LEDs 940.

The plurality of LEDs 940 for treatment may be arranged along five lines. Each line may be located below the fingers. Accordingly, the plurality of LEDs 940 for treatment may effectively apply light to the user's fingers. In addition, the plurality of LEDs 940 for treatment may be arranged concentrically. Therefore, the plurality of LEDs 940 for treatment may effectively apply light to the user s palm.

The first bio-signal measuring unit 550 or the third bio-signal measuring unit 3210 may further include a heating unit support 950. The heating unit support 950 may be fixed on the lower cover 920. The heating unit support 950 may be located below a heating unit to maintain the shape of the heating unit. The heating unit support 950 may have an upwardly convex shape. In a case in which the user naturally puts the hand on the first bio-signal measuring unit 550 or the third bio-signal measuring unit 3210, when viewed from the user's palm, the user's palm may be concave. Accordingly, the heating unit support 950 may be convex upward 902 to effectively transmit heat to the user's palm.

The heating unit support 950 may be arranged above the plurality of LEDs 940 for treatment. The heating unit support 950 may be made of a transparent material not to hide light of the LEDs 940.

The first bio-signal measuring unit 550 or the third bio-signal measuring unit 3210 may further include a heating unit 960. The heating unit 960 may be fixed on the heating unit support 950. The heating unit 960 may be formed along the convex surface of the heating unit support 950. The heating unit 960 may generate heat based on a control signal of the control unit 300. The heating unit 960 may provide thermal fomentation to the user.

The heating unit 960 may be located above the plurality of LEDs. The heating unit 960 may have a shape not to hide the light of the LEDs 940. For instance, the heating unit 960 may include at least one slit. Due to the slit, light of the LEDs 940 is not hidden by the heating unit 960, and can be transmitted to the user's palm.

FIG. 10 is a view for depicting a first bio-signal measuring unit according to an embodiment of the present disclosure.

The arm massage unit may include a cover sheet 1010. The cover sheet 1010 may be a component provided to complete the design of the massage apparatus 100. The cover sheet 1010 may be made of leather, artificial leather, or polyurethane (PU). Moreover, the cover sheet 1010 may be provided to fix the first bio-signal measuring unit 550.

Referring to FIGS. 9 and 10, the upper cover 970 may include an upper cover fixing portion 972. The upper cover fixing portion 972 may be formed along the circumference of the palm support 971 of the upper cover 970 and may be flat. The upper cover fixing portion 972 may include at least one hole or hooking portion. The hanging portion may have a hook shape. The at least one hole or hooking portion of the upper cover fixing portion 972 is coupled to a hooking portion or hole of the lower cover 920 so that the upper cover 970 may be fixed to the lower cover 920.

The cover sheet 1010 may cover at least a portion of the upper cover fixing portion 972. The at least a portion of the upper cover fixing portion 972 may be positioned under the cover sheet 1010 of the arm massage unit to prevent the upper cover 970 from being separated from the cover sheet 1010 of the arm massage unit. That is, the cover sheet 1010 can prevent the first bio-signal measuring unit 550 from moving horizontally or vertically. The first bio-signal measuring unit 550 can move only in the back-and-forth direction.

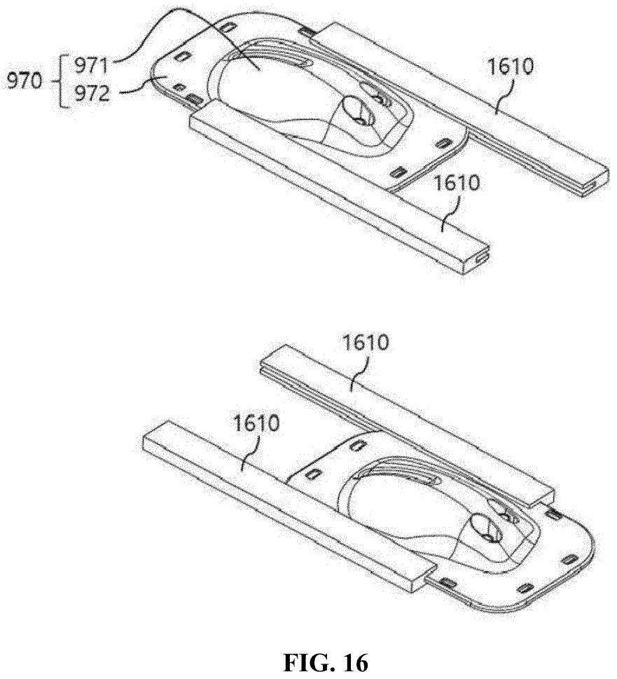

Although not illustrated in FIG. 9 or 10, guide rails may be formed on the plate 910 in the back-and-forth direction. The lower cover 920 may move back and forth along the guide rails. The guide rails may prevent the lower cover 920 from moving horizontally or vertically. Referring to FIG. 16, the plate 910 on which the guide rails are formed will be described.

FIG. 16 is a view for depicting a first bio-signal measuring unit according to an embodiment of the present disclosure.

The plate 910 according to an embodiment of the present disclosure may include guide rails 1610. The guide rails 1610 may be formed on the plate 910 in the back-and-forth direction. The guide rails 1610 may includes grooves extending back and forth. The left side and the right side of the upper cover fixing portion 972 may be inserted into the grooves of the guide rails 1610. The first bio-signal measuring unit 550 may move back and forth along the guide rails 1610. The guide rails 1610 may prevent the upper cover 970 or the lower cover 920 from moving horizontally or vertically. That is, the guide rails 1610 may prevent the first bio-signal measuring unit 550 from moving horizontally or vertically.

According to an embodiment of the present disclosure, the first bio-signal measuring unit 550 may not include at least two magnets 930 since the first bio-signal measuring unit 550 can move back and forth by the guide rails 1610, but is not limited thereto. The first bio-signal measuring unit 550 may include magnets so that first bio-signal measuring unit 550 can firmly move on the plate 910 including the guide rails 1610.

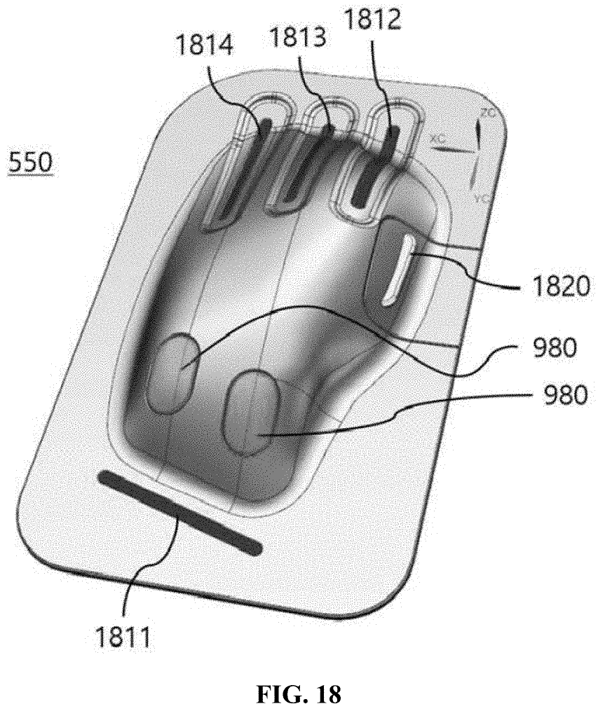

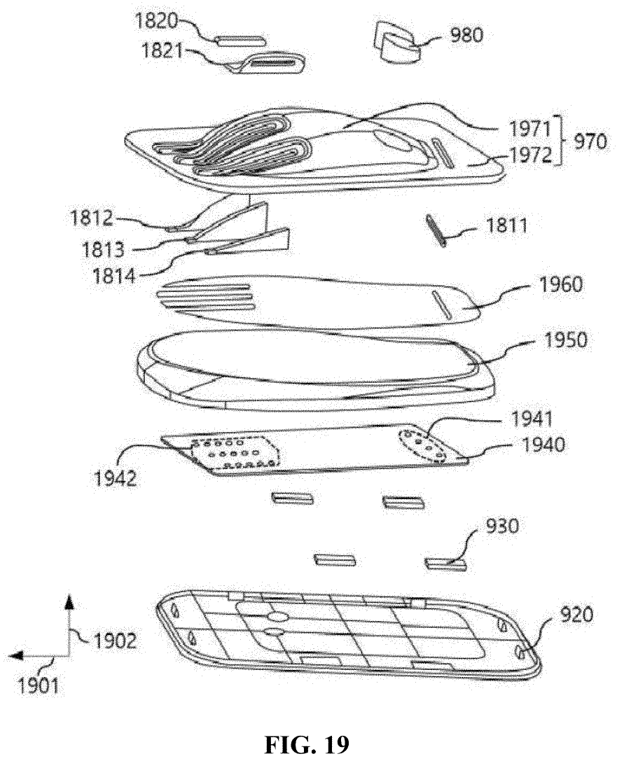

FIG. 18 is a perspective view of the first bio-signal measuring unit according to an embodiment of the present disclosure. FIG. 19 is a view for depicting components included in the first bio-signal measuring unit according to an embodiment of the present disclosure.

FIGS. 18 and 19 are views for depicting an embodiment different from the embodiment of FIGS. 8 and 9, but have components duplicating the components of FIGS. 8 and 9. In addition, the duplicated description thereof will be omitted. Therefore, undescribed contents with respect to FIGS. 18 and 19 can be understood with reference to FIGS. 8 and 9.

In FIG. 19, the front 1901 may mean a direction that the user is looking at when the user sits on the massage apparatus 100. In addition, the upper side 1902 may mean the upper side when the user sits on the massage apparatus 100.

Referring to FIGS. 18 and 19, the first bio-signal measuring unit 550 may include a function button 1820. The function button 1820 may be included in a user input unit 2180. The first bio-signal measuring unit 550 may include at least one function button 1820.

The function button 1820 may receive an input for a predetermined function. For example, the predetermined function may be a reclining on/off function or a massage mode start/stop function. However, the function button 1820 may not receive only an input for the predetermined function. The function of the function button 1820 may be set/changed by the control unit 300. The control unit 300 may change the function of the function button 1820 based on a user's input.

The first bio-signal measuring unit of the left side and the first bio-signal measuring unit of the right side may respectively include function buttons 1820. The function buttons 1820 included in the first bio-signal measuring unit of the left side and the first bio-signal measuring unit of the right side may perform different functions.

Referring to FIG. 19, the first bio-signal measuring unit 550 may include a function button guide 1821. The function button guide 1821 may be a structure for guiding the movement of the function button 1820.

The function button guide 1821 may be injection-molded separately from the upper cover 970. Additionally, the function button guide 1821 may be coupled to the upper cover 970, but is not limited thereto, and may be formed integrally with the upper cover 970.

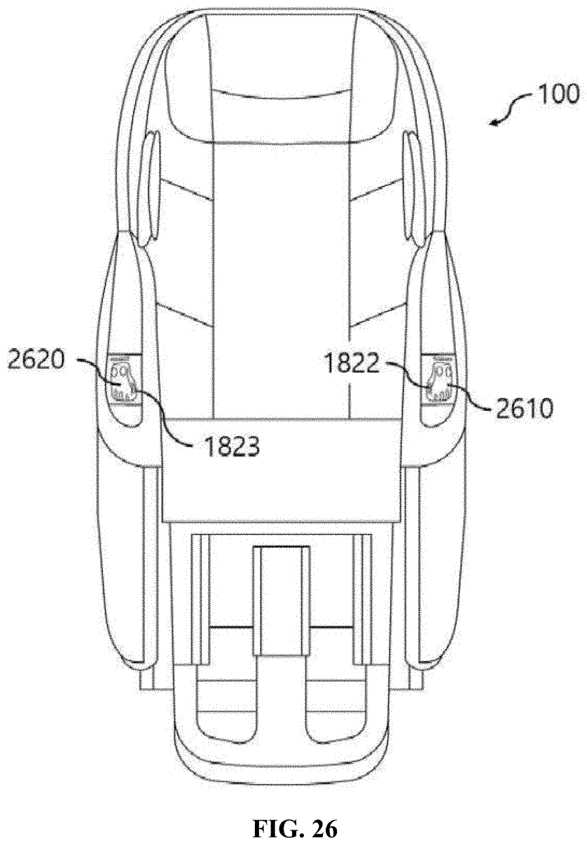

FIG. 26 is a view for depicting a function of a function button according to an embodiment of the present disclosure.

Referring to FIG. 26, the massage apparatus 100 may include a first bio-signal measuring unit 2610 of the left side arranged on a left armrest unit and a first bio-signal measuring unit 2620 of the right side arranged on a right armrest unit. The first bio-signal measuring unit 2610 of the left side may include a first function button 1822. Furthermore, the first bio-signal measuring unit 2620 of the right side may include a second function button 1823. The user can control the massage apparatus 100 by using the function buttons 1822 and 1823 arranged at the right and left sides even in a state in which both hands of the user are accommodated in the arm massage unit 500 to get a massage. That is, the user can easily manipulate various functions of the massage apparatus 100 even in a situation that the user is hard to move since the user's arms are grasped in the expanded arm airbags of the arm massage unit 500.

The massage apparatus 100 is configured for the user to set different functions of the function buttons 1822 and 1823 according to the current operation state of the massage apparatus 100. For instance, in a case in which the massage apparatus 100 is providing massage to the user, one of the function buttons 1822 and 1823 may be a button for stopping the provision of massage. However, in a case in which the massage apparatus 100 is not providing massage, one of the function buttons 1822 and 1823 may be changed into a button for executing provision of massage. As described above, since the function defined to the function button may be changed when the operation state of the massage apparatus is changed, the massage apparatus 100 according to the present disclosure can control various functions suitable for the current situation by using a small number of buttons.

According to an embodiment of the present disclosure, in a case in which the user presses one of the function buttons 1822 and 1823, the massage apparatus 100 may change an angle of the chair. More specifically, in a case in which the user presses the first function button 1822, the massage apparatus 100 may change an angle of the chair. The massage apparatus 100 may continuously change the angle while the user presses the first function button 1822. Moreover, the massage apparatus 100 may move at a predetermined angle whenever the user presses the first function button 1822. The massage apparatus 100 may repeat the operation that gradually lays down the chair and gradually makes the chair stand when the user presses the first function button 1822. The user can stop the angle control by pulling the hand away from the first function button 1822 when the chair reaches a desired angle.

According to an embodiment of the present disclosure, in a case in which the user presses the other function button in a state in which the massage apparatus 100 stops, the massage apparatus 100 may start to provide massage to the user based on a recommended massage mode. The recommended massage mode may be previously stored in the massage apparatus 100. Here, the other function button may be the second function button 1823. In addition, in a case in which the user presses the other button in a state in which the massage apparatus 100 is moving, the massage apparatus 100 may temporarily stop the massage mode. The massage apparatus 100 may temporarily stop all operations thereof, but is not limited thereto. The massage apparatus 100 may stops only some operations. For instance, the massage apparatus 100 may temporarily stop only the expansion/contraction of the arm airbag, but may continuously perform other massage operations. In a case in which the user presses the other button in a state in which the massage apparatus 100 is temporarily stopped, the massage apparatus 100 may restart the massage operation. For instance, if the entire operations of the massage apparatus are temporarily stopped, the massage apparatus 100 may restart the entire operations. Alternatively, if only some operations of the massage apparatus 100 are temporarily stopped, the massage apparatus 100 may restart the some operations. For example, if only the arm airbag operation is temporarily stopped, the massage apparatus 100 may restart the arm airbag operation.

In addition, according to an embodiment of the present disclosure, in a case in which the user presses any one among the function buttons 1822 and 1823, the massage mode may be ended. That is, the massage apparatus 100 may not provide massage to the user. Additionally, the massage apparatus 100 may return to the basic state.

Till now, the predetermined operations of the function buttons 1822 and 1823 have been described. However, the present disclosure is not limited thereto. The massage apparatus 100 can set functions of the function buttons 1822 and 1823 based on the use's selection. For instance, the massage apparatus 100 may display a menu for setting the function buttons 1822 and 1823 on a display. The massage apparatus 100 may receive the user's input to show which operation will be executed according to patterns that the user presses the function buttons 1822 and 1823. The pressed pattern of the function buttons 1822 and 1823 may include “a case in which the first function button 1822 and the second function button 1823 are simultaneously pressed shortly once”, “a case in which the first function button 1822 and the second function button 1823 are simultaneously pressed shortly twice”, or “a case in which the first function button 1822 and the second function button 1823 are simultaneously pressed long”. Moreover, The pressed pattern of the function buttons 1822 and 1823 may include a case in which the first function button 1822 is pressed shortly and the second function button 1823 is pressed shortly in a fixed time. Based on the received input by the user, the massage apparatus 100 may determine which operation will be executed when the function buttons 1822 and 1823 are pressed in the specific pattern by the user.

Referring to FIGS. 18 and 19, the first bio-signal measuring unit 550 may include light guides 1811, 1812, 1813 and 1814. The light guides 1811, 1812, 1813 and 1814 may have a configuration to transmit light emitted from the plurality of LEDs 1941 and 1942 to the user's hand.

The light guides 1811, 1812, 1813 and 1814 may be made of a transparent material. Moreover, the light guides 1811, 1812, 1813 and 1814 may be made of a material that selectively transmits the wavelength of the light emitted from the plurality of LEDs 1941 and 1942.

The light guide 1811 may be arranged on the wrist. Due to the light guide 1811, the user can prevent tunnel syndrome by applying the light emitted from the LEDs 1941 to the wrist. Furthermore, the light guides 1811, 1812, 1813 and 1814 may be arranged on a portion of the user's fingers. Due to the light guides 1811, 1812, 1813 and 1814, the user may prevent diseases which may be caused to finger joints by applying the light emitted from the plurality of LEDs 1942 for treatment.

Referring to FIG. 19, the upper cover 970 may include a palm support 1971 and an upper cover fixing portion 1972. The palm support 1971 may have holes for inserting the light guides 1812, 1813 and 1814 thereinto. Moreover, the upper cover fixing portion 1972 may have a hole for inserting the light guide 1811 thereinto.

The first bio-signal measuring unit 550 may further include a heating unit support 1950. The heating unit support may be fixed on the lower cover 920. The heating unit support 1950 may be located below a heating unit to maintain the shape of the heating unit. The heating unit support 1950 may have an upwardly convex shape. In a case in which the user naturally puts the hand on the first bio-signal measuring unit 550, when viewed from the user's palm, the user's palm may be concave. Accordingly, the heating unit support 1950 may be convex upward 1902 to effectively transmit heat to the user's palm. In addition, as illustrated in FIG. 19, the upper surface of the heating unit support 1950 may be flat.

The heating unit support 1950 may be arranged above the plurality of LEDs 1941 and 1942 for treatment. The heating unit support 1950 may be made of a transparent material not to hide light of the LEDs 1941 and 1942.

The first bio-signal measuring unit 550 may further include a heating unit 1960. The heating unit 1960 may be fixed on the heating unit support 1950. The heating unit 1960 may be put on the flat portion of the upper surface of the heating unit support 1950. The heating unit 1960 may generate heat based on a control signal of the control unit 300. The heating unit 1960 may provide thermal fomentation to the user.

The heating unit 1960 may be located above the plurality of LEDs 1902. The heating unit 1960 may have a shape not to hide the light of the LEDs 1941 and 1942. For instance, the heating unit 1960 may include at least one slit. Due to the slit, light of the LEDs 1941 and 1942 is not blocked by the heating unit 960, and can be transmitted to the user's palm. In addition, the at least one slit included in the heating unit 1960 may be arranged at lower ends of the light guides 1811, 1812, 1813 and 1814. Referring to FIG. 19, the heating unit 1960 may have three slits formed in the back-and-forth direction for the light guides 1811, 1812, 1813 and 1814. The light of the LEDs 1942 may be transmitted to the user's palm due to the three slits formed in the back-and-forth direction. Moreover, the heating unit 1960 may have one slit formed in the horizontal direction for the light guide 1811. Due to the slit formed in the horizontal direction, the light of the plurality of LEDs 1941 may be transmitted to the user's wrist.

The first bio-signal measuring unit 550 may include a plurality of LEDs 1941 and 1942 for treatment. The plurality of LEDs 1941 and 1942 may be fixed on the lower cover 920. The plurality of LEDs 1941 and 1942 for treatment may emit light based on a control signal of the control unit 300. The plurality of LEDs 1941 and 1942 for treatment may have a configuration to apply LED light to the user's wrist. Additionally, the plurality of LEDs 1941 and 1942 for treatment may have a configuration to apply LED light to the user's fingers.

The plurality of LEDs 1941 and 1942 for treatment may be located on an LED control PCB 1940. The LED control PCB 1940 may include a processor and a memory. The control unit 300 may send a control signal to the LED control PCB 1940. The LED control PCB 1940 may turn on or off at least a portion of the LEDs 1941 and 1942 based on the control signal.

The first bio-signal measuring unit 550 may include a lower cover 920. The lower cover 920 may be placed on the housing of the arm massage unit. The lower cover 920 may be fixed on the housing of the arm massage unit, but is not limited thereto. For example, the lower cover 920 may not be fixed on the housing of the arm massage unit. The lower cover 920 may be slidable on the housing of the arm massage unit.

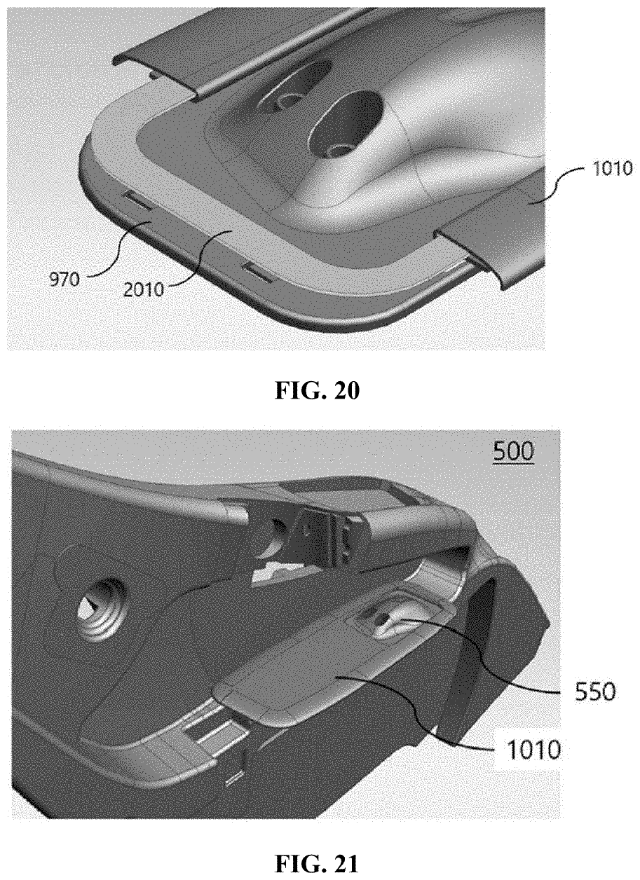

FIG. 20 is a view for depicting a first bio-signal measuring unit according to an embodiment of the present disclosure. FIG. 21 is a view for depicting a first bio-signal measuring unit according to an embodiment of the present disclosure.

Although FIG. 20 illustrates an embodiment different from that of FIG. 9, some of components of FIG. 20 may be duplicated with those of FIG. 9. FIG. 20 may be the same as FIGS. 18 and 19. Components which are not described in conjunction with FIG. 20 may be described in conjunction with FIG. 9.

FIG. 20 illustrates that a portion of a cover sheet 1010 is controlled for the convenience of description. Additionally, FIG. 20 does not illustrate an electrode inserted into the upper cover 970.

In FIG. 20, a plate 2010 may be disposed on the upper cover 970. The plate may be fixed on the cover sheet 1010 of the arm massage unit 500. That is, at least a portion of the plate 2010 may be surrounded by the cover sheet 1010. Moreover, the whole of the plate 2010 may be surrounded by the cover sheet 1010. FIG. 20 illustrates that a portion of the plate 2010 is not surrounded by the cover sheet 1010, but it is because a portion of the cover sheet 1010 is not illustrated for the convenience of description.

The plate 2010 may be located on the upper cover 970. More specifically, the plate 2010 may be located on an upper cover fixing portion 1972 of the upper cover 970. The plate 2010 may be arranged along the upper cover fixing portion 1972. A portion of the cover sheet 1010 may be located between the upper cover 970 and the plate 2010. The plate 2010 may be made of a metal material. At least two magnets 930 may face the plate 2010.

Referring to FIGS. 19 and 20, the magnets 930 located below the upper cover 970 may generate a magnetic force with the plate 2010. The plate 2010 and the upper cover 970 may come into contact with each other by the magnetic force between the magnets 930 and the plate 2010. In addition, due to the magnetic force between the magnets 930 and the plate 2010, it is prevented that the cover sheet 1010 surrounding the plate 2010 is separated from the plate 2010. Therefore, according to the present disclosure, it is prevented that the cover sheet 1010 becomes crumpled.

Due to the magnetic force between the magnets 930 and the plate 2010, the first bio-signal measuring unit 550 may not move horizontally. Furthermore, due to the cover sheet 1010, the first bio-signal measuring unit 550 may not move horizontally and vertically. FIG. 21 illustrates an example in which the first bio-signal measuring unit 550 is located below the cover sheet 1010 according to FIG. 20.

FIG. 11 illustrates a second bio-signal measuring unit according to an embodiment of the present disclosure.

The second bio-signal measuring unit 610 may include a leg support 1110. Referring to FIGS. 6 and 11, the leg support 1110 may be formed on the housing of the foot massage unit 622 to be located at the back of the leg of the user. Here, the back may mean the back side when the user sits on the massage apparatus 100.

The second bio-signal measuring unit 610 may include a foot holder 1150. The foot holder 1150 may be coupled to the front of the leg support 1110. The foot holder 1150 may support the user's leg. Furthermore, the foot holder 1150 may have a concave shape toward the user so that the user can feel comfortable when putting the leg on the foot holder 1150.

The second bio-signal measuring unit 610 may include an elastic layer 1160. The elastic layer 1160 may be attached to the front of the foot holder 1150. The elastic layer 1160 may be made of an elastic material. Due to the elastic layer 1160, the user may feel comfortable even if the user's leg touches the electrode.

The second bio-signal measuring unit 610 may include a cover layer 1170. The cover layer 1170 may be attached on the front of the elastic layer 1160. The cover layer 1170 may be a component for providing the user with a clean design. The cover layer 1170 may be made of leather, artificial leather or polyurethane.

The second bio-signal measuring unit 610 may include a second bio-signal measuring electrode 118. The second bio-signal measuring electrode 1180 may be inserted into a hole formed in the front of the cover layer. In addition, the second bio-signal measuring electrode 1180 may be a silicon electrode.

The control unit 300 may measure the user's bio-signal by using at least one among the first bio-signal measuring electrode 980 and the second bio-signal measuring electrode 610. The second bio-signal measuring electrode 1180 may get in contact with the user. The second bio-signal measuring electrode may be electrically connected to the control unit 300. The control unit 300 may acquire a bio-signal based on a signal from the second bio-signal measuring electrode 1180.

The second bio-signal measuring electrode 1180 may connect an electric wire from the second bio-signal measuring electrode 1180 to the control unit 300 to be electrically connected with the control unit 300. The leg support 1110, the foot holder 1150, the elastic layer 1160, and a cover layer 1170 may respectively have holes for wiring. The user may put the leg on the bio-signal measuring unit 610. In this instance, the foot holder 1150, the elastic layer 1160, and the cover layer 1170 may move forward and backward with respect to the leg support 1110. Due to the movement, an electric wire holder of an elastic material (not illustrated) may hold the electric wire so that the electric wire between the second bio-signal measuring electrode 1180 and the control unit 300 is not damaged. The electric wire holder may be fixed at any one among the foot holder 1150, the elastic layer 1160, and the cover layer 1170. The electric wire holder may enhance durability of the massage apparatus 100.

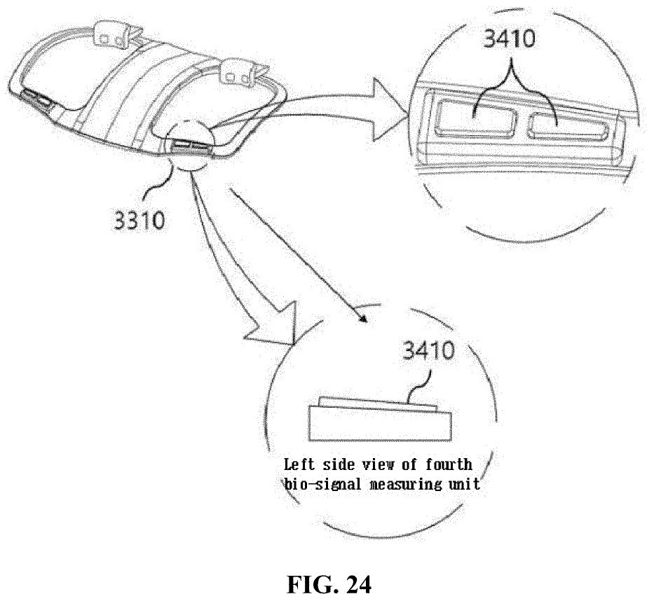

Referring to FIG. 24, the fourth bio-signal measuring unit 3310 may include a fourth bio-signal measuring electrode 3410. FIG. 24 is a left side view of the bio-signal measuring unit 3310. The upper surface of the fourth bio-signal measuring electrode 3410 faces the user, and is at an angle of 1 to 45 degrees to the ground surface. Specifically, in a case in which the leg massage unit 2300 of the massage apparatus 100 is in the basic state, the upper surface of the fourth bio-signal measuring electrode 3410 faces the user, and is at an angle of 1 to 45 degrees to the ground surface. The basic state is a posture of the massage apparatus 100 when the massage apparatus 100 does not provide massage, and may mean a state in which the user can easily sit on the massage apparatus 100. Furthermore, the basic state may be a state in which the leg massage unit 2300 is nearly perpendicular to the ground surface.

In addition, the fourth bio-signal measuring electrode 3410 may be a silicon electrode. The control unit 300 may measure the user's bio-signal by using at least one among the third bio-signal measuring electrode 3220, the second bio-signal measuring electrode 1180, and the fourth bio-signal measuring electrode 3410. The fourth bio-signal measuring electrode 3410 may get in contact with the user. The fourth bio-signal measuring electrode 3410 may be electrically connected with the control unit 300. The control unit 300 may acquire the bio-signal based on the signal from the fourth bio-signal measuring electrode 3410. In order to electrically connect the fourth bio-signal measuring electrode 3410 to the control unit 300, an electric wire may be connected from the fourth bio-signal measuring electrode 3410 to the control unit 300.

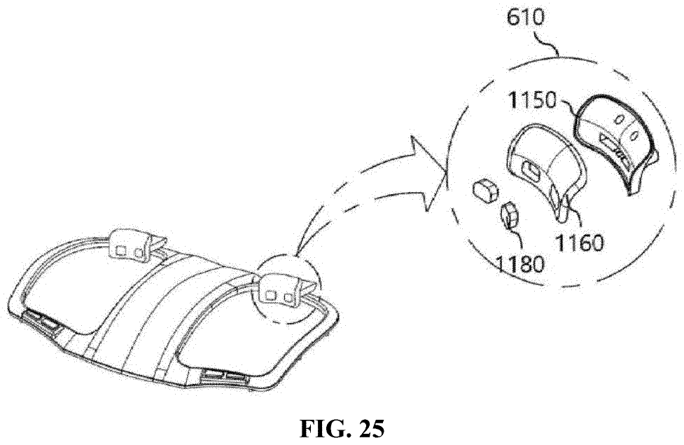

FIG. 25 illustrates a second bio-signal measuring unit according to an embodiment of the present disclosure.

FIG. 25 illustrates a portion of the components of the second bio-signal measuring unit 610. Components which are not described in conjunction with FIG. 25 may be described in conjunction with FIG. 11.

According to various embodiments of the present disclosure, the second bio-signal measuring unit 610 may include a foot holder 1150. The foot holder 1150 may be coupled to the front of the leg support 1110. The foot holder 1150 may support the user's leg. In addition, the foot holder 1150 may have a concave shape toward the user so that the user feels comfortable when the user puts the foot on the foot holder 1150. Additionally, the foot holder 1150 may be injection-molded with silicon. Since the foot holder 1150 is made of a soft material, the user may feel comfortable even when the second bio-signal measuring unit 610 touches the user's foot.

The second bio-signal measuring unit 610 may include an elastic layer 1160. The elastic layer 1160 may be attached to the front of the foot holder 1150. The elastic layer 1160 and the foot holder 1150 may be bonded to each other using double sided tape. The elastic layer 1160 may be made of an elastic material. For instance, the elastic layer 1160 may be made of a silicon material. Moreover, the elastic layer 1160 may have a hardness of HS40 or less. Even if the user's leg touches the electrode, the user may feel softness due to the elastic layer 1160.

The second bio-signal measuring unit 610 may not include the cover layer 1170 in order to prevent the cover layer 1170 from interfering between the user's foot and the electrode.

The second bio-signal measuring unit 610 may include a second bio-signal measuring electrode 1180. The second bio-signal measuring electrode 1180 may be inserted into a hole formed in the elastic layer 1160. In addition, the second bio-signal measuring electrode 1180 may be a silicon electrode.

The second bio-signal measuring electrode 1180 may have a thickness of 5.0 to 7.0 mm. In addition, the horizontal and vertical lengths of the second bio-signal measuring electrode 1180 may range from 13 mm to 17 mm. The second bio-signal measuring electrode 1180 having such a size can most accurately measure a bio-signal.

The second bio-signal measuring electrode 1180 may be connected to a cable by an internal snap button. The control unit 300 may measure the user's bio-signal using the second bio-signal measuring electrode 1180.

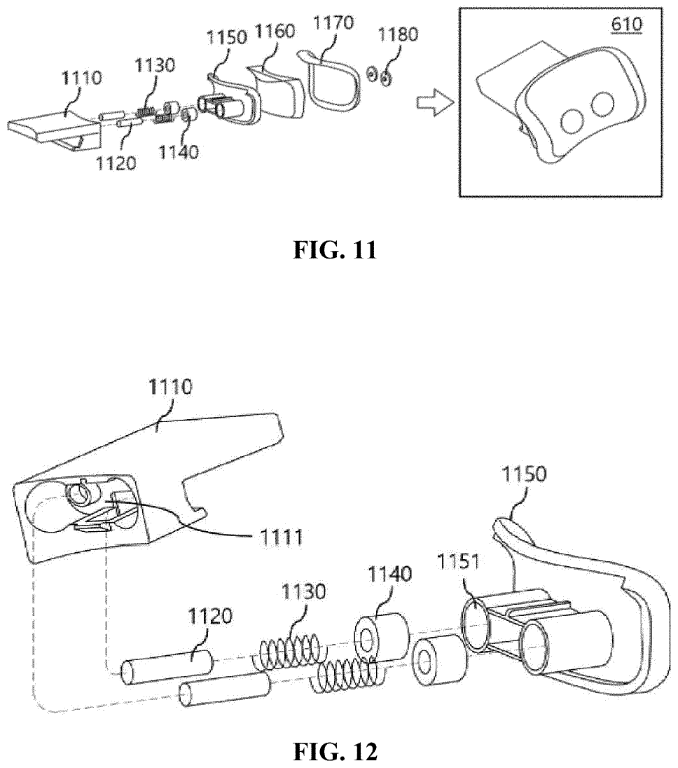

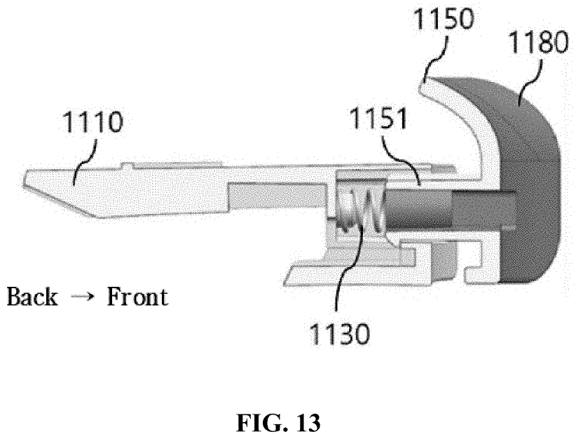

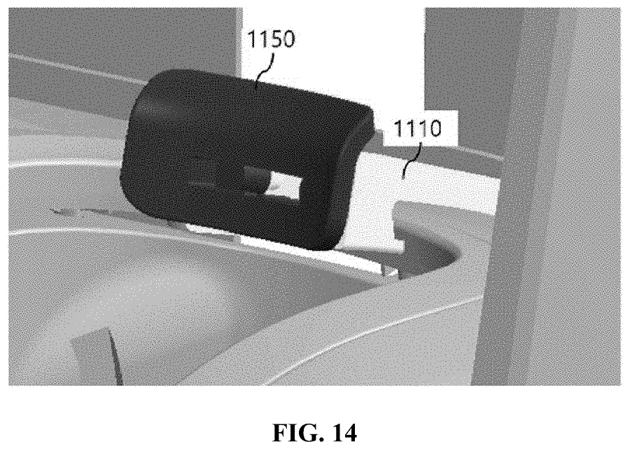

FIG. 12 is a view for depicting a second bio-signal measuring unit according to an embodiment of the present disclosure. FIG. 13 is a view for depicting a second bio-signal measuring unit according to an embodiment of the present disclosure. FIG. 14 is a view for depicting a second bio-signal measuring unit according to an embodiment of the present disclosure.

Referring to FIGS. 12 to 14, the foot holder 1150 may include a coupling protrusion 1151. The coupling protrusion 1151 may be inserted into a coupling groove 1111 of the leg support 1110. The coupling protrusion 1151 may have a groove. A guide ring 1140 may be inserted into the groove of the coupling protrusion 1151.

A guide pin 1120 may be inserted into a spring 1130. Moreover, the guide pin 1120 may be inserted into the groove of the guide ring 1140. The spring 1130 may not be inserted into the groove of the guide ring 1140. The spring 1130 may receive a force by one side of the guide ring 1140.

The guide pin 1120, the spring 1130, the guide ring 1140, and the coupling protrusion 1151 of the foot holder 1150 may be inserted into the coupling groove 1111 of the leg support 1110. The spring 1130 may receive a force by one side of the coupling groove 111 and one side of the guide ring 1140 to be compressed. The spring 1130 may push the guide ring 1140 forward. Therefore, the second bio-signal measuring electrode 1180 may easily touch the back of the leg of the user.

The massage apparatus 100 may include two first bio-signal measuring units 550. For instance, the massage apparatus 100 may include a first bio-signal measuring unit of the left side and a first bio-signal measuring unit of the right side. Each of the first bio-signal measuring unit of the left side and the first bio-signal measuring unit of the right side may include two first bio-signal measuring electrodes.

The massage apparatus 100 may include two second bio-signal measuring units 610. For instance, the massage apparatus 100 may include a second bio-signal measuring unit of the left side and a second bio-signal measuring unit of the right side. Each of the second bio-signal measuring unit of the left side and the second bio-signal measuring unit of the right side may include two second bio-signal measuring electrodes.

The massage apparatus 100 may include two third bio-signal measuring units 3210. For instance, the massage apparatus 100 may include a third bio-signal measuring unit of the left side and a third bio-signal measuring unit of the right side. Each of the third bio-signal measuring unit of the left side and the third bio-signal measuring unit of the right side may include two third bio-signal measuring electrodes.

The massage apparatus 100 may include two fourth bio-signal measuring units 3310. For instance, the massage apparatus 100 may include a fourth bio-signal measuring unit of the left side and a fourth bio-signal measuring unit of the right side. Each of the fourth bio-signal measuring unit of the left side and the fourth bio-signal measuring unit of the right side may include two fourth bio-signal measuring electrodes.

The control unit 300 may measure the user's bio-signal by using at least two among the four first bio-signal measuring electrodes, at least two among the four second bio-signal measuring electrodes, at least two among the four third bio-signal measuring electrodes, and at least two among the four fourth bio-signal measuring electrodes. Therefore, the massage apparatus 100 may accurately measure a bio-signal. In addition, the control unit 300 may acquire information on the user's body condition based on the measured bio-signal. A general bio-signal measurer may ask a user to take a special posture because the measurer cannot properly measure a bio-signal if the user's arms touch the body or the legs touch each other. However, in a case in which the user sits or lies on the massage apparatus 100 according to the present disclosure, the user can take the optimum posture to measure a bio-signal due to the housing of the massage apparatus 100. Therefore, the massage apparatus 100 according to the present disclosure can measure a bio-signal the most accurately.

In addition, the massage apparatus 100 may periodically measure a bio-signal while providing massage. Accordingly, the user's health improvement effect according to the massage can be displayed. Moreover, the massage apparatus 100 may cumulatively store the measured bio-signals of the user or information on the body condition, or may transfer the measured bio-signals or information on the body condition to a cloud server. The measured bio-signals of the user or information on the body condition may be used for diagnosis of the user later.

Furthermore, the massage apparatus 100 can acquire information on a plurality of bio-signals or a plurality of body conditions by periodically performing measurement while providing massage once. Providing massage once may mean that the massage apparatus 100 provides one massage mode to the user. The massage apparatus 100 may acquire information on a representative bio-signal or a representative body condition out of information on the plurality of bio-signals or the plurality of body conditions. For instance, the massage apparatus 100 may acquire at least one among an average value, a median value, a variance, or a standard deviation of information on the plurality of bio-signals or the plurality of body conditions, and may use the acquired value as information on the representative bio-signal or the representative body condition. Accordingly, the user can easily understand the characteristics of the bio-signal or the characteristics of the information on the body condition by number of times of massages.

In addition, the massage apparatus 100 may represent bio-signals over time or information on body conditions on a display in a graph on the basis of information on a plurality of bio-signals or a plurality of body conditions. The massage apparatus 100 may represent information on a representative bio-signal or representative body condition according to the number of massages on a display in a graph on the basis of a plurality of bio-signals or information on a plurality of body conditions.

FIG. 24 is a view illustrating a fourth bio-signal measuring unit 3310 according to an embodiment of the present disclosure.

The control unit 300 may measure the user's bio-signal by using at least one among the first bio-signal measuring electrode 980, the second bio-signal measuring electrode 1180, the third bio-signal measuring electrode 3220, and the fourth bio-signal measuring electrode 3410. The fourth bio-signal measuring electrode 3410 may come into contact with the user. The fourth bio-signal measuring electrode 3410 may be electrically connected to the control unit 300. In order to electrically connect the fourth bio-signal measuring electrode 3410 to the control unit 300, an electric wire may be connected from the fourth bio-signal measuring electrode 3410 to the control unit 300. Based on the signal from the fourth bio-signal measuring electrode 3410, the control unit 300 may obtain a bio-signal.

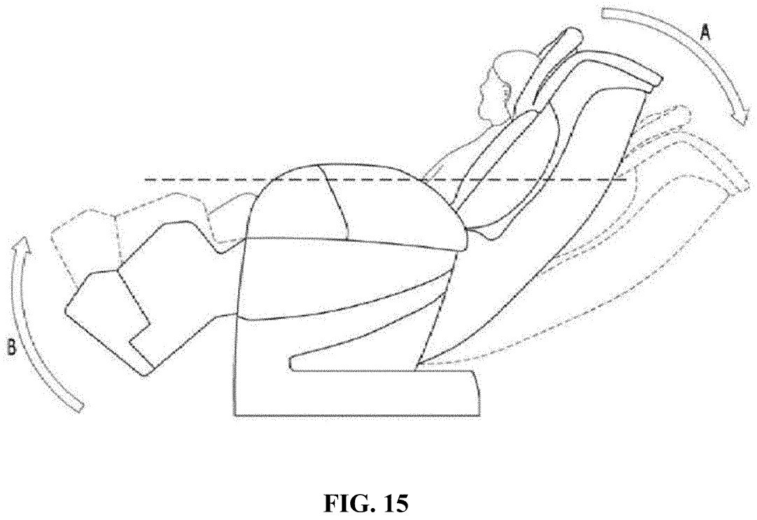

FIG. 15 is a view for depicting an example in which a posture of the massage apparatus is adjusted to measure a bio-signal according to an embodiment of the present disclosure.

Referring to FIG. 15, the control unit 300 may control the operation of at least one among the first bio-signal measuring unit 550, the second bio-signal measuring unit 610, the third bio-signal measuring unit 3210, and the fourth bio-signal measuring unit 3310. The control unit 300 may perform bio-signal measurement after adjusting the posture of the massage apparatus 100 in a case in which the user inputs measurement of information on the body condition.

Moreover, the massage apparatus 100 may automatically measure the user's bio-signal. While the massage apparatus 100 provides massage to the user according to the massage mode, the control unit 300 may measure the user's bio-signal at a predetermined cycle by using at least two among the first bio-signal measuring unit 550, the second bio-signal measuring unit 610, the third bio-signal measuring unit 3210, and the fourth bio-signal measuring unit 3310. Furthermore, the control unit 300 may periodically obtain information on the user's body condition based on the measured bio-signal. The massage apparatus 100 may adjust the posture of the massage apparatus 100 according to the massage mode. In a case in which the posture of the massage apparatus 100 is suitable for measuring bio-signals, the control unit 300 may measure the user's bio-signal by using at least two among the first bio-signal measuring unit 550, the second bio-signal measuring unit 610, the third bio-signal measuring unit 3210, and the fourth bio-signal measuring unit 3310. If the user is sitting or lying on the massage apparatus 100, it may always be a suitable posture for measuring a bio-signal, so the control unit 300 may periodically measure bio-signals while the massage apparatus 100 provides massage. In addition, the control unit 300 may periodically obtain information on the user's body condition based on the bio-signal. The information on the body condition may includes at least one among body composition information and electrocardiogram information.

The control unit 300 may simultaneously measure body composition information and electrocardiogram information of the user by using at least two among the first bio-signal measuring unit 550, the second bio-signal measuring unit 610, the third bio-signal measuring unit 3210, and the fourth bio-signal measuring unit 3310. Signals for obtaining body composition information and electrocardiogram information may be similar signals. The control unit 300 may simultaneously measure body composition information and electrocardiogram information of the user by analyzing signals received from at least two among the first bio-signal measuring unit 550, the second bio-signal measuring unit 610, the third bio-signal measuring unit 3210, and the fourth bio-signal measuring unit 3310. It may take about 15 seconds for the massage apparatus 100 to measure body composition by using at least one among the first bio-signal measuring unit 550, the second bio-signal measuring unit 610, the third bio-signal measuring unit 3210, and the fourth bio-signal measuring unit 3310. In addition, it may take about one minute for the massage apparatus 100 to measure electrocardiogram by using at least one among the first bio-signal measuring unit 550, the second bio-signal measuring unit 610, the third bio-signal measuring unit 3210, and the fourth bio-signal measuring unit 3310. The control unit 300 may simultaneously measure body composition information and electrocardiogram information by using at least a portion of the plurality of electrodes included in at least one among the first bio-signal measuring unit 550, the second bio-signal measuring unit 610, the third bio-signal measuring unit 3210, and the fourth bio-signal measuring unit 3310.

However, the present disclosure is not limited thereto. The control unit 300 may measure at least one among body composition information and electrocardiogram information at a predetermined cycle by using at least one among the first bio-signal measuring unit 550, the second bio-signal measuring unit 610, the third bio-signal measuring unit 3210, and the fourth bio-signal measuring unit 3310. The cycle for obtaining body composition information and the cycle for obtaining electrocardiogram information may be different from each other or may be the same.

The control unit 300 may cumulatively store the measured bio-signals of the user and/or information on the body condition of the user. Moreover, the control unit 300 may transmit the bio-signals of the user and/or information on the body condition of the user to a cloud server and/or a hospital server with the user's approval. Accordingly, the user can check the process of changing the body condition of the user. In addition, when a medical person later diagnoses the user's health condition, the cumulatively stored bio-signals and/or information on the body condition may be used. Therefore, the user's health condition can be accurately diagnosed.

### First Embodiment of Posture Adjustment of Massage Apparatus 100

According to an embodiment of the present disclosure, the massage apparatus 100 may measure a bio-signal in a state in which the leg massage unit 2300 of the massage apparatus 100 is not lifted. The state in which the leg massage unit 2300 is not lifted means a state in which the angle between the leg massage unit 2300 and the vertical line of the ground is less than a predetermined angle. Furthermore, the massage apparatus 100 may measure a bio-signal in a state in which the body massage unit 2100 of the massage apparatus 100 is not tilted. The state in which the body massage unit 2100 is not tilted may mean a state in which the user is sitting on the massage apparatus 100 with the waist erected. Even if the leg massage unit 2300 is not lifted, when the user puts the sole of the foot on the fourth bio-signal measuring unit 3310 located on the upper surface of the housing of the leg massage unit, the fourth bio-signal measuring unit 3310 comes into close contact with the user's sole so that the bio-signal may be measured more accurately. In addition, according to an embodiment of the present disclosure, the massage apparatus 100 may measure a bio-signal without tilting the body massage unit 2100 backward. In a case in which the body massage unit 2100 is not tilted backward and the leg massage unit 2300 is not lifted, the massage apparatus 100 may measure a bio-signal from the user's palm by using the first bio-signal measuring unit 550 and/or the second bio-signal measuring unit 3210, and may measure a bio-signal from the user's sole of the foot by using the fourth bio-signal measuring unit.

### Second Embodiment of Posture Adjustment of Massage Apparatus 100

According to an embodiment of the present disclosure, the massage apparatus 100 may measure a bio-signal after the leg massage unit 2300 of the massage apparatus 100 is lifted (B). In the second embodiment, the angle between the leg massage unit 2300 and the vertical line of the ground may be more than a predetermined angle. For instance, the massage apparatus 100 may operate the leg angle actuator provided in the body massage unit 2100 to lift the leg massage unit 2300, and the control unit 300 measure a bio-signal after the leg massage unit 2300 is lifted. In a case in which the massage apparatus 100 lifts the leg massage unit 2300 and the angle between the leg massage unit 2300 and the vertical line of the ground is more than a predetermined angle, the second bio-signal measuring unit 610 located at the back of the user's leg comes into close contact with the user's leg so that the bio-signal can be more accurately measured. That is, in a case in which the massage apparatus 100 does not tilt the body massage unit 2100 backward and lifts the leg massage unit 2300, the massage apparatus 100 may measure a bio-signal from the user's hand by using the first bio-signal measuring unit 550 and/or the second bio-signal measuring unit 3210, and may measure a bio-signal from the user's heel by using the second bio-signal measuring unit 610.

### Third Embodiment of Posture Adjustment of Massage Apparatus 100

According to an embodiment of the present disclosure, the massage apparatus 100 may measure a bio-signal after tilting the body massage unit 2100 backward (A). For instance, the massage apparatus 100 may operate the back angle actuator included in the body massage unit 2100 to tilt the body massage unit 2100 backward, and the control unit 300 may measure a bio-signal after tilting the body massage unit 2100 backward. In a case in which the body massage unit 2100 is tilted backward, the user's hand may move backward. As described above, the first bio-signal measuring unit 550 may move forward and backward. Accordingly, in a case in which the body massage unit 2100 is tilted backward, the first bio-signal measuring unit 550 moves backward, so that the first bio-signal measuring unit 550 is located below the user's palm. Therefore, the massage apparatus 100 may measuring the user's bio-signal accurately. In addition, the third bio-signal measuring unit 3210 is fixed on the arm massage unit, but may come into contact with the user's palm even if the body massage unit 2100 is tilted backward since the third bio-signal measuring unit 3210 has a horizontally long electrode. Therefore, the massage apparatus 100 may accurately measure the user's bio-signal. Additionally, in the third embodiment, the leg massage unit 2300 may not be lifted. As described above, in this case, when the user puts the sole on the fourth bio-signal measuring unit 3310 located on the upper surface of the housing of the leg massage unit, the fourth bio-signal measuring unit 3310 comes into contact with the user's sole so that the bio-signal may be accurately measured. To sum up, in the case in which the body massage unit 2100 is tilted backward and the leg massage unit 2300 is not lifted, the massage apparatus 100 may measure a bio-signal from the user's palm by using the first bio-signal measuring unit 550 and/or the second bio-signal measuring unit 3210, and may measure a bio-signal from the user's sole by using the fourth bio-signal measuring unit 3310.

### Fourth Embodiment of Posture Adjustment of Massage Apparatus 100

According to an embodiment of the present disclosure, the massage apparatus 100 may lift the leg massage unit 2300 of the massage apparatus 100 (B), and measure a bio-signal after tilting the body massage unit 2100 backward (A). For instance, the massage apparatus 100 may operate the leg angle actuator included in the body massage unit 2100 to lift the leg massage unit 2300, operate the back angle actuator to tilt the body massage unit 2100 backward, and then, measure a bio-signal. In the case in which the body massage unit 2100 is tilted backward and the leg massage unit 230 is lifted, the massage apparatus 100 may measure a bio-signal from the user's palm by using the first bio-signal measuring unit 550 and/or the second bio-signal measuring unit 3210, and may measure a bio-signal from the user's heel by using the fourth bio-signal measuring unit 3310.

According to an embodiment of the present disclosure, the massage apparatus 100 may determine whether to measure a bio-signal based on the massage mode. The user may do not want that the user's biometric information is acquired unnecessarily. The user may select a massage mode not to acquire the bio-signal so that the massage apparatus 100 does not measure the bio-signal.

The control unit 300 may periodically measure bio-signals from at least one among the first bio-signal measuring unit 550, the second bio-signal measuring unit 610, the third bio-signal measuring unit 3210, and the fourth bio-signal measuring unit 3310. The control unit 300 may determine an electrode which does not get in contact with the user's skin based on the signal received from at least one among the first bio-signal measuring unit 550, the second bio-signal measuring unit 610, the third bio-signal measuring unit 3210, and the fourth bio-signal measuring unit 3310. In a case in which there is an electrode determined as the electrode which does not get in contact with the user's skin, the control unit 300 may generate an alarm signal. The alarm signal may include information on the location of the electrode which does not get in contact with the user's skin. The control unit 300 can control the alarm signal to be output through the output unit. The alarm signal may be output into light or sound. The user can adjust the posture based on the alarm signal so that the skin comes into contact with the electrode.

In a case in which it is determined that a portion of the electrode included in at least one among the first bio-signal measuring unit 550, the second bio-signal measuring unit 610, the third bio-signal measuring unit 3210, and the fourth bio-signal measuring unit 3310 does not get in contact with the user's skin, the control unit 300 may control to measure a bio-signal just by the electrode getting in contact with the skin. More specifically, in a case in which it is determined that a portion of the electrode included in at least one among the first bio-signal measuring unit 550, the second bio-signal measuring unit 610, the third bio-signal measuring unit 3210, and the fourth bio-signal measuring unit 3310 does not get in contact with the user's skin, the control unit 300 may output an alarm signal, and wait for a predetermined period of time till a portion of the electrode included in at least one among the first bio-signal measuring unit 550, the second bio-signal measuring unit 610, the third bio-signal measuring unit 3210, and the fourth bio-signal measuring unit 3310 gets in contact with the user's skin. Even after the predetermined period of time, if it is determined that a portion of the electrode included in at least one among the first bio-signal measuring unit 550, the second bio-signal measuring unit 610, the third bio-signal measuring unit 3210, and the fourth bio-signal measuring unit 3310 does not get in contact with the user's skin, the control unit 300 may control to measure a bio-signal just by the electrode getting in contact with the skin. Even though the user does not see or hear the alarm signal, the massage apparatus 100 may accurately measure the bio-signal and obtain information on the user's body condition. The control unit 300 may store the information on the user's body condition in the memory. In addition, the control unit 300 may store also information on the position of the used electrode in the memory in order to obtain information on the user's body condition.

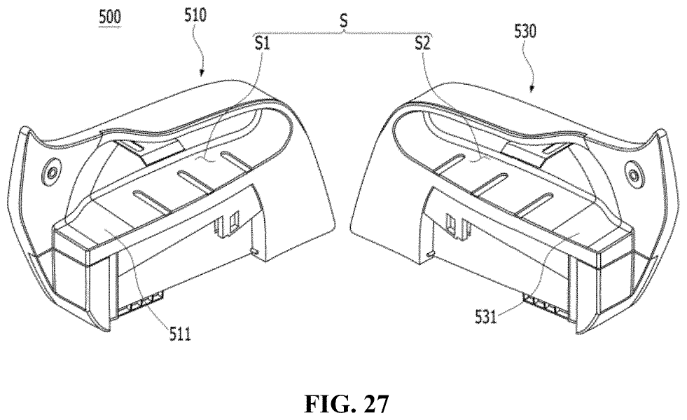

FIG. 27 is a view for depicting an arm massage unit according to an embodiment of the present disclosure.

The arm massage unit 500 may include a space S for accommodating the user's arm.

For instance, the arm massage unit 500 may include first and second arm massage units 510 and 530 as described above.

The first arm massage unit 510 may include a first arm massage unit frame 511 (or a first arm massage unit housing), and a first space S1 formed in the first arm massage unit frame 511. For instance, the first arm massage unit frame 511 may be formed in a “⊂” shape which is opened at one side. The first space S1 is formed on the ⊂-shaped inner wall of the first arm massage unit frame 511 to accommodate one arm of the user.

The second arm massage unit 531 may include a second arm massage unit frame 531 (or a second arm massage unit housing), and a second space S2 formed in the second arm massage unit frame 531. For instance, the second arm massage unit frame 531 may be formed in a “⊃” shape which is opened at one side. The second space S2 is formed on the ⊃-shaped inner wall of the second arm massage unit frame 531 to accommodate one arm of the user.

As described above, the first arm massage unit frame 511 is formed in the “⊂” shape and the second arm massage unit 531 is formed in the “⊃” shape, but the present disclosure is not limited thereto. For instance, the first arm massage unit frame 511 and the second arm massage unit 531 may be formed in various shapes, for example, in a shape of which facing sides are opened. Hereinafter, for convenience of description, it is assumed that the first arm massage unit frame 511 is formed in the “⊂” shape and the second arm massage unit 531 is formed in the “⊃” shape.

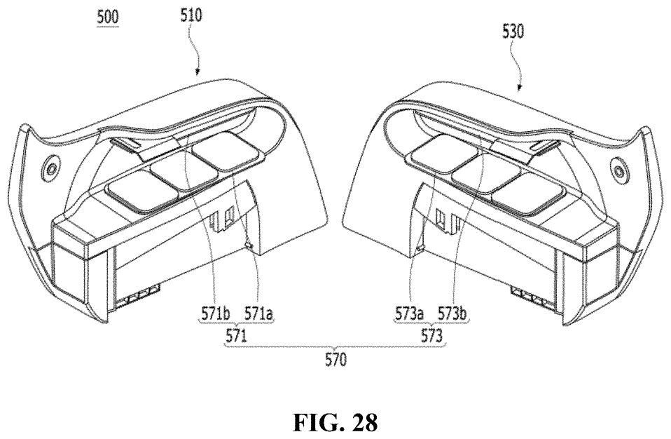

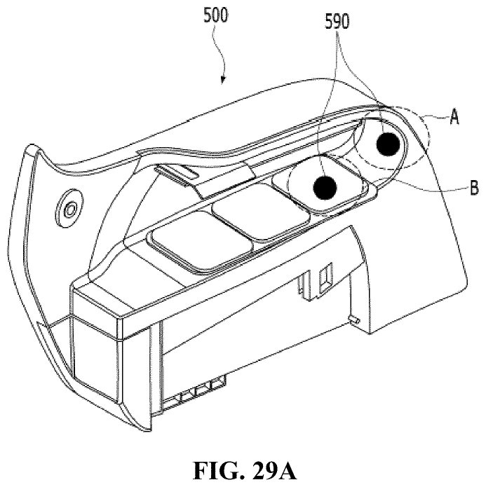

FIG. 28 is a view for depicting an arm air cell according to an embodiment of the present disclosure.

The arm massage unit 500 may include an arm air cell 570 arranged in the space S. The arm air cell 570 may massage the user's arm by expanding and contracting based on the control of the control unit 300.

For example, the arm air cell 570 may include a first arm air cell 571 included in the first arm massage unit 510, and a second arm air cell 573 included in the second arm massage unit 530.

The first arm air cell 571 is arranged in the first space S1 to massage one arm of the user. For instance, the first arm air cell 571 may include at least one among a first upper air cell 571a and a first lower air cell 571b. The first upper air cell 571a may be arranged on the top surface of the ⊂-shaped inner wall of the first arm massage unit frame 511. The second lower air cell 573b may be arranged on the bottom surface of the ⊂-shaped inner wall of the first arm massage unit frame 511.

The second arm air cell 573 is arranged in the second space S2 to massage the other arm of the user. For instance, the second arm air cell 573 may include at least one among a second upper air cell 573a and a second lower air cell 573b. The second upper air cell 573a may be arranged on the top surface of the ⊃-shaped inner wall of the second arm massage unit frame 531. The second lower air cell 573b may be arranged on the bottom surface of the ⊃-shaped inner wall of the second arm massage unit frame 531.

The control unit 300 may acquire pneumatic pressure of the air cell 570. For example, the massage apparatus 100 may include a pneumatic pressure sensor for sensing pneumatic pressure of the air cell 570. The pneumatic pressure sensor may be disposed on at least one among the air cell 570, an airline connected to the air cell 570, and an air supply unit supplying air to the air cell 570. The pneumatic pressure sensor senses pneumatic pressure of the air cell 570 to transmit pneumatic pressure of the air cell 570 to the control unit 300.

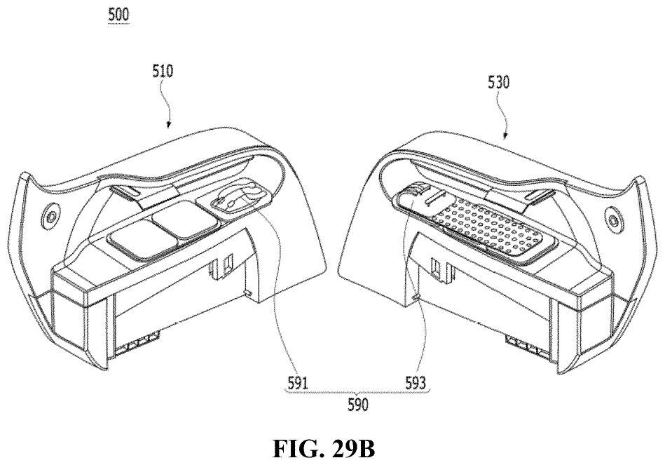

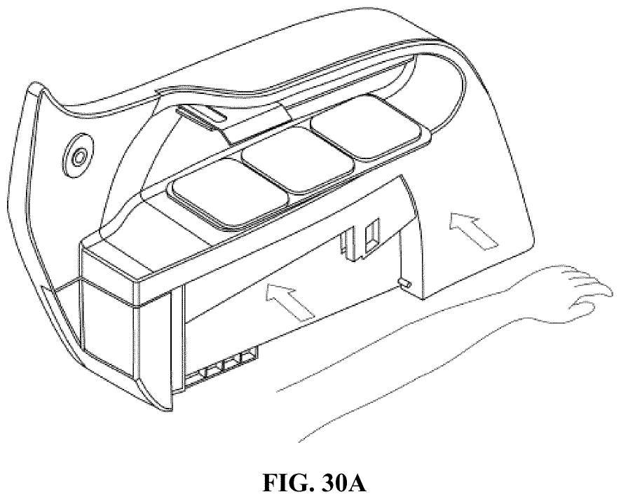

FIGS. 29A-29B are views for depicting a sensing unit according to an embodiment of the present disclosure. Specifically, FIG. 29A illustrates a location of a sensing unit according to an embodiment of the present disclosure, and FIG. 29B illustrates an example of a sensing unit according to an embodiment of the present disclosure.

Referring to FIG. 29A, the arm massage unit 500 may include a sensing unit 590 arranged in the space S. The sensing unit may sense the user's arm based on the control of the control unit 300. For example, the sensing unit 590 may sense the user's arm accommodated in the space S to generate a sensing signal, and transmit the generated sensing signal to the control unit 300. The sensing signal may indicate that the user's arm is accommodated in the space S.

The sensing unit 590 may be at least one among an imaging device, a pressure sensor, an optical sensor, and a bio-signal sensor. In this instance, the imaging device may be various imaging devices, such as a thermal imaging camera and an infrared camera. The bio-signal sensor may be at least one among the first bio-signal measuring unit 550 and the third bio-signal measuring unit 3210 described above. However, as described above, the sensing unit 590 may be at least one among an imaging device (or a camera), a pressure sensor, an optical sensor, and a bio-signal sensor, but is not limited thereto, and may be various devices capable of sensing the arm.

As illustrated in FIG. 28A, the sensing unit 590 may be arranged in at least a region of the entire area of the arm massage unit 500. For instance, the sensing unit 590 may be disposed in at least one among a region A and a region B. In this instance, the region A may be a region corresponding to one end direction of the arm massage unit 500 (or a region corresponding to the forward direction of the massage apparatus 100, the front surface of the inner walls of the arm massage unit frames 511 and 531) out of the entire inner wall area of the arm massage unit frames 511 and 531. The region B may be a region corresponding to the user's palm (or a lower end region of the inner wall) out of the entire area of the inner walls of the arm massage unit frames 511 and 531.

As described above, the sensing unit 590 is arranged in at least one among the regions A and B, but is not limited thereto. For instance, the sensing unit 590 may be arranged in various regions, for example, an upper surface area of the inner wall, different from the regions A and B out of the entire area of the inner walls of the arm massage unit frames 511 and 531.

Referring to FIG. 29B, the sensing unit 590 may includes a first sensing unit 591, and a second sensing unit 593 included in the second arm massage unit 530.

The first sensing unit 591 may be disposed in the first space S1. For instance, the first sensing unit 591 may be disposed in at least a portion (for instance, the region A, the region B, etc.), out of the entire area of the inner wall of the first arm massage unit frame 511.

The first sensing unit 591 may sense one arm of the user. For instance, the first sensing unit 591 senses one arm of the user accommodated in the first space S1 to generate a first sensing signal, and transmit the first sensing signal to the control unit 300. The first sensing signal may indicate that one arm of the user was accommodated in the first space S1.

The second sensing unit 593 may be disposed in the second space S2. For instance, the second sensing unit 593 may be disposed in at least a portion (for instance, the region A, the region B, etc.), out of the entire area of the inner wall of the second arm massage unit frame 531. The second sensing unit 593 may sense the other arm of the user. For instance, the second sensing unit 593 senses the other arm of the user to generate a second sensing signal, and transmit the second sensing signal to the control unit 300. The second sensing signal may indicate that the other arm of the user was accommodated in the first space S1.

The first and second sensing units 591 and 593 may be implemented by at least one among the first and third bio-signal measuring units 550 and 3210.

As illustrated in FIG. 27B, in a case in which at least one among the first and second sensing units 591 and 593 is implemented by the first bio-signal measuring unit 550, the at least one sensing unit may be disposed on the top surface of the lower air cell arranged on the top surface of the arm massage unit frame as described with respect to the third bio-signal measuring unit 3210. The at least one sensing unit may be indirectly connected to the arm massage unit frame through the lower air cell.

The sensing unit which is implemented by the at least one among the first and third bio-signal measuring units 550 and 3210 may include at least one among the first bio-signal measuring electrode 980 and the third bio-signal measuring electrode 3320, and the sensing unit may transmit an electrical value of the electrode to the control unit 300 as a sensing signal.

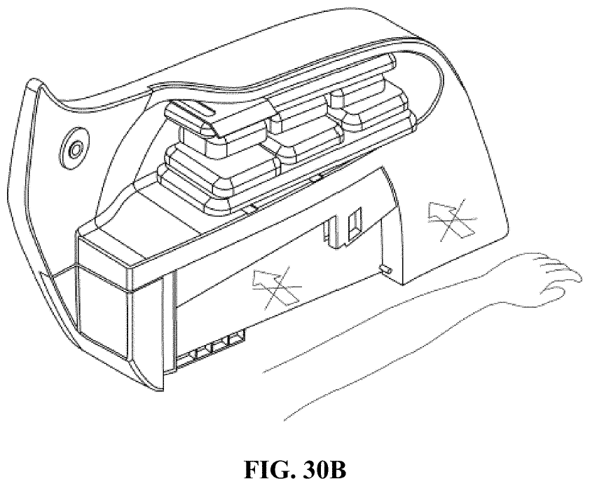

FIGS. 30A-30B are views for depicting a state of the arm air cell according to an embodiment of the present disclosure. Specifically, FIG. 30A is a view for depicting a contracted state of the arm air cell according to an embodiment of the present disclosure, and FIG. 30B is a view for depicting an expanded state of the arm air cell according to an embodiment of the present disclosure.

Referring to FIGS. 30A and 30B, the arm massage unit 500 may expand and contract the arm air cell 570 to massage the user's arm. In this instance, the expansion and contraction of the arm air cell 570 may be performed repeatedly.

In a case in which the arm air cell 570 is contracted as illustrated in FIG. 30A, the user's arm may freely move inward or outward from the arm massage unit 500. However, in a case in which the arm massage unit 500 is operated in a state in which the user's arm is moved outward from the arm massage unit 500, the arm massage unit 500 performs an action to massage the user's arm even though the arm massage unit 500 cannot massage the user's arm, thereby wasting power of the arm massage unit 500.

In addition, in a case in which the arm air cell 570 is expanded as illustrated in FIG. 30B, the user's arm cannot freely move inward or outward from the arm massage unit 500. However, in a case in which the user moves the arm inward or outward from the arm massage unit 500 in the state in which the arm air cell 570 is expanded, the expanded arm air cell 570 hinders the movement of the arm of the user so that the user cannot freely use the arm, thereby lowering user satisfaction with the massage apparatus 100.

In other words, the arm massage unit 500 may cause a problem of wasting power due to the operation of massaging the user's arm even if the user's arm is not located inside the arm massage unit 500 in the state in which the arm massage unit 500 is operating. Moreover, since the user cannot freely use the arm in the state in which the arm air cell 570 is expanded, user satisfaction with the massage apparatus 100 gets lower.

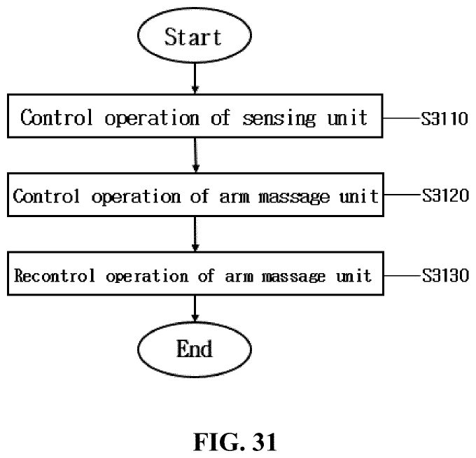

FIG. 31 is a view for depicting an arm massage unit control operation of the control unit according to an embodiment of the present disclosure.

According to an embodiment of the present disclosure, the control unit 300 may control the operation of the sensing unit 590 in a sensing section, and control the operation of the arm massage unit 500 in an operating section. Accordingly, the control unit 300 may start an arm massage by operating the arm massage unit 500 or may suspend and/or end the arm massage by stopping the arm massage unit 500 according to whether the user's arm is sensed by the sensing unit 590.

First, the control unit 300 may control the operation of the sensing unit 590 in the sensing section (S3110). For instance, the control unit 300 may activate the sensing unit 590 by operating the sensing unit 590 in the sensing section. The activated sensing unit 590 may transmit a sensing signal to the control unit 300 after sensing the user's arm. In this instance, the sensing section may be a section to sense the user's arm. The sensing unit 590 may be various devices for sensing the user's arm as described above. The control unit 300 may repeatedly operate and stop the sensing unit 590 based on a predetermined time cycle.

The operation state of the arm massage unit 500 in the sensing section may be at least one among an operation state and a stop state. The operation state may be a state in which the arm massage unit 500 is operating (or operated). The stop state may be a state in which the arm massage unit 500 is stopped (or is not operated).

Thereafter, the control unit 300 may control the operation of the arm massage unit 500 in the operating section based on the sensing signal of the sensing unit 590 (S3120). For instance, the control unit 300 may maintain or change the operation of the arm massage unit 500 based on the sensing signal. In this instance, the operating section may be a section to control the operation of the arm massage unit 500. The sensing unit 500 is in an activated state in the operating section, but is not limited thereto. For instance, the sensing unit 500 may be inactivated in the operating section.

Finally, the control unit 300 may recontrol the operation of the arm massage unit 500 based on pneumatic pressure of the air cell 570 (S3130).

Hereinafter, steps S3120 and S3130 will be described in detail.

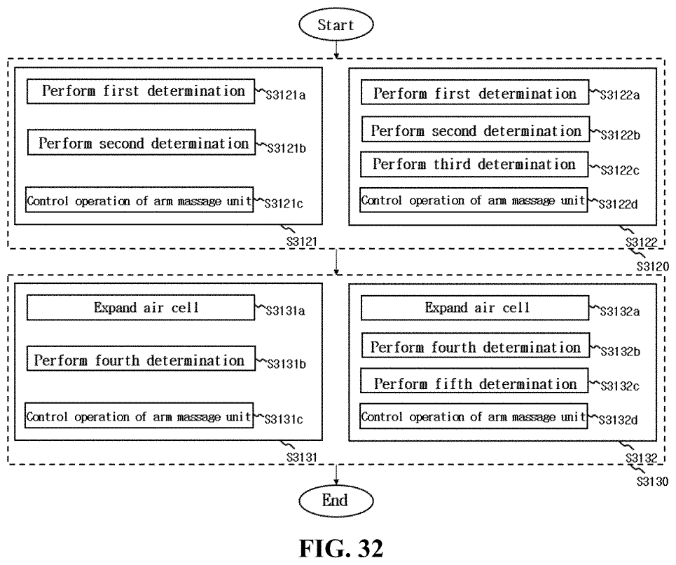

FIG. 32 is a view for depicting an arm massage unit control operation illustrated in FIG. 31.

According to an embodiment of the present disclosure, the control unit 300 may control the operation of the arm massage unit 500 based on the sensing signal of the sensing unit 590 and the operation state of the arm massage unit 500 (S3120).

For instance, the control unit 300 may determine whether the sensing signal transmitted from the sensing unit 590 is received, and control the operation of the arm massage unit 500 based on the determination result and the operation state of the arm massage unit 500 (S3121).

For instance, the control unit 300 may perform a first determination to determine whether the sensing signal transmitted from the sensing unit 590 is received (S3121a). When the sensing unit 590 senses the user's arm, the sensing unit 590 transmits the sensing signal to the control unit 300, and the control unit 300 receives the sensing signal.

When the sensing signal is received, the control unit 300 may determine that the user's arm is accommodated in the space S. When the sensing signal is not received, the control unit 300 may determine that the user's arm is not accommodated in the space S.

The control unit 300 may perform a second determination to determine whether the operation state of the arm massage unit 500 is in an operating state (S3121b). For instance, the control unit 300 may obtain various kinds of information about the arm massage unit 500, such as power supply of the arm massage unit 500, air supply of the arm air cell 570, and the like. The control unit 300 may determine whether the operation state of the arm massage unit 500 is in the operating state based on the obtained information.

The control unit 300 may control the operation of the arm massage unit 500 based on the first and second determination results (S3121c).

In a case in which the sensing signal is not received and the operation state is a stop state, or in a case in which the sensing signal is received and the operation state is the operating state, the control unit 300 may maintain the operation of the arm massage unit 500 in the operating section. For example, the control unit 300 may maintain the operated state of the arm massage unit 500 in a case in which the operation state is in the operating state, and maintain the arm massage unit 500 in a stopped state in a case in which the operation state is the stop state.

In a case in which the sensing signal is not received and the operation state is the operating state, the control unit 300 may change the operation of the arm massage unit 500 by stopping the arm massage unit 300.

In a case in which the sensing signal is received and the operation state is the stop state, the control unit 300 may change the operation of the arm massage unit 500 by operating the arm massage unit 300.

As described above, the control unit 300 may determine whether the user's arm is accommodated in the space S through the sensing signal and control the operation of the arm massage unit 500.

In another example, the control unit 300 may control the operation of the arm massage unit 500 based on a sensing signal, an operation state of the arm massage unit 500, and receiving time of the sensing signal (S3122). In this instance, the receiving time may range from the moment the control unit 300 receives the sensing signal transmitted from the sensing unit 590 to the moment the sensing signal is not received. The receiving time may be determined based on sensing time that the sensing unit 590 senses the user's arm. For instance, the receiving time may be longer in a case in which the sensing time is long than in a case in which the sensing time is short. In other words, the receiving time may be increased as the sensing unit 590 senses the user's arm for a long time.

For instance, the control unit 300 may perform the first determination (S3122a), and then, perform the second determination (S3122b). Step S3122a is the same as the above-described step S3121a, and step S3122b is the same as the above-described step S3121b. Accordingly, detailed description thereof will be omitted.

In a case in which the sensing signal is received, the control unit 300 may perform a third determination to determine whether the receiving time of the sensing signal exceeds a predetermined threshold receiving time (S3122c). The threshold receiving time may be predetermined through at least one of various objects, such as an enterprise related with the massage apparatus 100, a manager who manages the massage apparatus 100, a user who uses the massage apparatus 100, and the like, and may be stored in the memory 330. The enterprise related with the massage apparatus 100 may be at least one among enterprises for manufacturing, production, distribution and selling of the massage apparatus 100. The threshold receiving time may be a standard to determine whether the user's arm is accommodated in the space S for enough time. In a case in which the user's arm is accommodated in the space S for enough time, the receiving time may exceed the threshold receiving time.

For example, the control unit 300 may compare the receiving time and the threshold receiving time to determine whether the receiving time exceeds the threshold receiving time. In a case in which the receiving time exceeds the threshold receiving time, the control unit 300 may determine that the user's arm is accommodated in the space S for enough time. In a case in which the receiving time is less than the threshold receiving time, the control unit 300 may determine that the user's arm is not accommodated in the space S for enough time. The control unit 300 can control the operation of the arm massage unit 500 based on the second and third determination results (3122d).

In at least one case among the case in which the operation state is the operating state and the receiving time exceeds the threshold receiving time and the case in which the operation state is the stop state and the receiving time is less than the threshold receiving time, the control unit 300 may maintain the operation of the arm massage unit 500.

In a case in which the operation state is the operating state and the receiving time is less than the threshold receiving time, the control unit 300 may change the operation of the arm massage unit 500 by stopping the arm massage unit 500.

In a case in which the operation state is the stop state and the receiving time exceeds the threshold receiving time, the control unit 300 may change the operation of the arm massage unit 500 by operating the arm massage unit 500.

As described above, the control unit 300 may control the operation of the arm massage unit 500 by determining whether the user's arm is accommodated in the space S for enough time through the receiving time of the sensing signal.

In addition, in a case in which the operation state of the arm massage unit 500 is changed into the operating state, the control unit 300 may recontrol the operation of the arm massage unit 500 (S3130). In the case in which the operation state of the arm massage unit 500 is changed into the operating state, the control unit 300 may control the operation of the arm massage unit 500 based on the pneumatic pressure of the air cell 570.

For example, the control unit 300 may control the operation of the arm massage unit 500 based on whether the pneumatic pressure of the air cell 570 exceeds a predetermined threshold pneumatic pressure (S3131). The threshold pneumatic pressure is predetermined through one of the various objects as described above, and then, is stored in the memory 330. The threshold pneumatic pressure may be a standard for determining whether the user's arm is exactly accommodated in the space S. In a case in which the user's arm is exactly accommodated in the space S, the pneumatic pressure of the expanded air cell 570 may exceed the threshold pneumatic pressure.

For instance, the control unit 300 may expand the air cell 570 (S3131a). The control unit 300 may operate the air supply unit to expand the air cell 570 by supplying air to the air cell 570.

The control unit 300 may perform a fourth determination to determine whether the pneumatic pressure of the expanded air cell 570 exceeds the threshold pneumatic pressure (S3131b).

For example, the control unit 300 may compare pneumatic pressure of the expanded air cell 570 and the threshold pneumatic pressure to determine whether the pneumatic pressure of the expanded air cell 570 exceeds the threshold pneumatic pressure.

In the case in which the pneumatic pressure of the expanded air cell 570 exceeds the threshold pneumatic pressure, the control unit 300 may determine that the user's arm is exactly accommodated in the space S. In the case in which the pneumatic pressure of the expanded air cell 570 is less than the threshold pneumatic pressure, the control unit 300 may determine that the user's arm is not exactly accommodated in the space S.

The control unit 300 may control the operation of the arm massage unit 500 based on the fourth determination result (S3131c).

In the case in which the pneumatic pressure of the expanded air cell 570 exceeds the threshold pneumatic pressure, the control unit 300 may maintain the operation of the arm massage unit 500. In other words, the control unit 300 may maintain the arm massage unit 500 in the operating state.

In the case in which the pneumatic pressure of the expanded air cell 570 is less than the threshold pneumatic pressure, the control unit 300 may change the operation of the arm massage unit by stopping the arm massage unit 500. In other words, the control unit 300 may change the operation state of the arm massage unit 500 into the stop state.

As described above, the control unit 300 may control the operation of the arm massage unit 500 by determining whether the user's arm is exactly accommodated in the space S through pneumatic pressure of the air cell.

In another embodiment, the control unit 300 may control the operation of the arm massage unit 500 based on pneumatic pressure of the air cell 570 and time of re-receiving time of the sensing signal (S3132).

For example, the control unit 300 may expand the air cell 570 (S3132a), and perform the fourth determination (S3132b). Step S3132a is the same as step S3131a, and step S3132b is the same as step S3131b. Accordingly, detailed description thereof will be omitted.

In the case in which the pneumatic pressure of the expanded air cell 570 exceeds the threshold pneumatic pressure, the control unit 300 may perform a fifth determination to determine whether the re-receiving time of the sensing signal exceeds predetermined threshold re-receiving time (S3132c). In this instance, the re-receiving time may be receiving time of the sensing signal transmitted to the control unit 300 after the air cell 570 is expanded. The re-receiving time may be determined based on sensing time that the sensing unit 590 senses the user's arm after the air cell 590 is expanded. For instance, the re-receiving time may be longer in a case in which the sensing time after expansion of the air cell 570 is long than in a case in which the sensing time after expansion of the air cell 570 is short. In other words, the re-receiving time may be increased as the sensing unit 590 after expansion of the air cell 570 senses the user's arm for a long time.

The threshold re-receiving time may be predetermined through at least one of various objects, and may be stored in the memory 330. The threshold re-receiving time may be a standard to determine whether the user's arm is accommodated in the space S for enough time after the air cell 570 is expanded. In the case in which the user's arm is accommodated in the space S for enough time after the air cell 570 is expanded, the re-receiving time may exceed the threshold re-receiving time.

The threshold receiving time and the threshold re-receiving time may be the same or different. Preferably, the threshold re-receiving time may be shorter than the threshold receiving time, but is not limited thereto. For instance, the threshold re-receiving time may be longer than the threshold receiving time.

For example, the control unit 300 may compare the re-receiving time and the threshold re-receiving time to determine whether the re-receiving time exceeds the threshold re-receiving time.

In the case in which the re-receiving time exceeds the threshold re-receiving time, the control unit 300 may determine that the user's arm is exactly accommodated in the space S for enough time. In a case in which the re-receiving time is less than the threshold re-receiving time, the control unit 300 may determine that the user's arm is not exactly accommodated in the space S for enough time.

The control unit 300 may control the operation of the arm massage unit 500 based on the fifth determination result (S3132d).

In the case in which the re-receiving time exceeds the threshold re-receiving time, the control unit 300 may maintain the operation of the arm massage unit 500. In other words, the control unit 300 may maintain the arm massage unit 500 in the operating state.

In a case in which the re-receiving time is less than the threshold re-receiving time, the control unit 300 may change the operation of the arm massage unit 500 by stopping the arm massage unit 500. In other words, the control unit 300 may change the operating state of the arm massage unit 500 into the stop state.

As described above, through the pneumatic pressure and re-receiving time of the air cell 570, the control unit 300 may determine whether the user's arm is exactly accommodated in the space S for enough time and whether the user's arm is accommodated in the space S for enough time after the air cell is expanded, and control the operation of the arm massage unit 500.

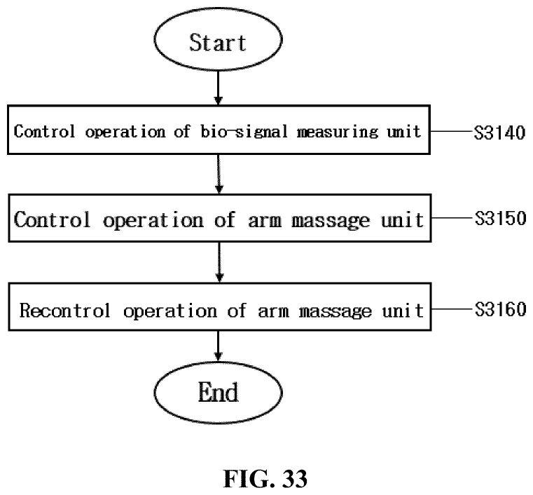

FIG. 33 illustrates an arm massage unit control operation of the control unit according to an embodiment of the present disclosure.

According to an embodiment of the present disclosure, the arm massage unit 500 may include electrodes arranged in the space. For instance, the sensing unit 590 may be implemented by any one measuring unit among the first and third bio-signal measuring units 550 and 3210. The any one measuring unit may include any one electrode among the first bio-signal measuring electrode 980 and the third bio-signal measuring electrode 3320. In other words, an electrode arranged in the space may be any one among the first bio-signal measuring electrode 980 and the third bio-signal measuring electrode 3320. Hereinafter, for convenience of description, it is assumed that the sensing unit 590 is implemented by the first bio-signal measuring unit 550.

According to an embodiment of the present disclosure, the control unit 300 may control the operation of the first bio-signal measuring unit 550 in the sensing section, and control the operation of the arm massage unit 500 based on an electrical value of the first bio-signal measuring unit 980 in the operating section.

For example, the control unit 300 may control the operation of the first bio-signal measuring unit 550 in the sensing section (S3140). For instance, the control unit 300 may activate the first bio-signal measuring unit 550 by operating the first bio-signal measuring unit 550 in the sensing section. The activated first bio-signal measuring unit 550 may transmit information on the electrical value of the first bio-signal measuring electrode 980 to the control unit 300. The electrical value of the first bio-signal measuring electrode 980 may be at least one among voltage, current, resistance and impedance of the first bio-signal measuring electrode 980. The control unit 300 may repeatedly operate and stop the first bio-signal measuring unit 550 based on a predetermined time cycle.

As described above, the first bio-signal measuring unit 550 transmits the electrical value of the first bio-signal measuring electrode 980, but is not limited thereto. For instance, the first bio-signal measuring unit 550 and the control unit 300 may be electrically connected to each other, and the control unit 300 may obtain an electrical value of the first bio-signal measuring electrode 980.

The control unit 300 may control the operation of the arm massage unit 500 in the operating section based on the electrical value of the first bio-signal measuring electrode 980 (S3150).

The control unit 300 may recontrol the operation of the arm massage unit 500 based on pneumatic pressure of the air cell 570 (S3160).

Hereinafter, steps S3150 and S3160 will be described in detail.

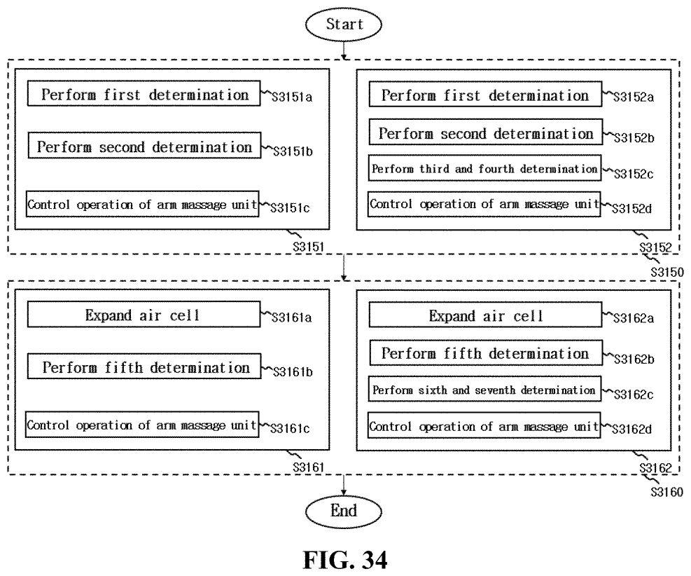

FIG. 34 is a view for depicting the arm massage unit control operation illustrated in FIG. 33.

According to an embodiment of the present disclosure, the control unit 300 may control the operation of the arm massage unit 500 based on the electrical value of the first bio-signal measuring electrode 980 and the operation state of the arm massage unit 500 (S3150).

For example, the control unit 300 may determine whether the electrical value of the first bio-signal measuring electrode 980 is changed, and control the operation of the arm massage unit 500 based on the determination result and the operation state of the arm massage unit 500 (S3151).

For instance, the control unit 300 may perform the first determination to determine whether the electrical value of the first bio-signal measuring electrode 980 is changed (S3151a). In a case in which the user's arm gets in contact with the first bio-signal measuring electrode 980, the electrical value of the first bio-signal measuring electrode 980 may be changed.

In a case in which the electrical value of the first bio-signal measuring electrode 980 is changed, the control unit 300 may determine that the user's arm gets in contact with the first bio-signal measuring electrode 980, and determine that the user's arm is accommodated in the space S. In a case in which the electrical value of the first bio-signal measuring electrode 980 is not changed, the control unit 300 may determine that the user's arm does not get in contact with the first bio-signal measuring electrode 980, and determine that the user's arm is not accommodated in the space S. The control unit 300 may perform a second determination to determine whether the arm massage unit 500 is in the operating state (S3151b).

The control unit 300 may control the operation of the arm massage unit 500 based on the first determination and the second determination results (S3151c).

In at least one among a case in which the electrical value of the first bio-signal measuring electrode 980 is not changed and the arm massage unit is in the stop state and a case in which the electrical value of the first bio-signal measuring electrode 980 is changed and the arm massage unit is in the operating state, the control unit 300 may maintain the operation of the arm massage unit 500.

In a case in which the electrical value of the first bio-signal measuring electrode 980 is not changed and the arm massage unit is in the operating state, the control unit 300 may stop the arm massage unit 500 to change the operation of the arm massage unit 500.

In a case in which the electrical value of the first bio-signal measuring electrode 980 is changed and the arm massage unit is in the stop state, the control unit 300 may operate the arm massage unit 500 to change the operation of the arm massage unit 500. As described above, the control unit 300 may determine whether the user's arm is not accommodated in the space S. through the electrical value of the first bio-signal measuring electrode 980 to control the operation of the arm massage unit 500.

In another embodiment, the control unit 300 may control the operation of the arm massage unit 500 based on at least one among the electrical value of the first bio-signal measuring electrode 980, the operation state of the arm massage unit 500, and a change amount and changing time with respect to the change amount (S3152).

The change amount may indicate the degree that the electrical value of the first bio-signal measuring electrode 980 is changed. The change amount may be determined based on at least one among a contact area between the user's arm and the first bio-signal measuring electrode 980 and the number of electrodes getting in contact with the user's arm. For example, the change amount may be greater when the contact area is large than when the contact area is small. The change amount may be greater when the number of electrodes getting in contact with the user's arm is large than when the number of electrodes getting in contact with the user's arm is small. In other words, the change amount may be increased as the contact area is large or the number of the electrodes getting in contact with the user's arm is large.

The changing time may range from the moment the electrical value of the first bio-signal measuring electrode 980 starts to change to the moment the electrical value of the first bio-signal measuring electrode 980 is not changed. The changing time may be determined based on contact time that the user's arm gets in contact with the first bio-signal measuring electrode 980. For instance, the changing time may be longer when the contact time is long than when the contact time is short. In other words, the changing time may be increased as the user's arm gets in contact with the first bio-signal measuring electrode 980 for a long period of time.

For example, the control unit 300 may perform the first determination to determine whether the electrical value is changed (S3152a), and perform the second determination to determine whether the operation state is in the operating state (S3152b). Step S3152a is the same as step S3151a, and step S3152b is the same as step S3151b. Accordingly, detailed description thereof will be omitted.

In a case in which the electrical value of the first bio-signal measuring electrode 980 is changed, the control unit 300 may perform at least one among the third determination to determine whether the change amount of the electrical value of the first bio-signal measuring electrode 980 exceeds a predetermined threshold change amount and the fourth determination to determine whether the changing time with respect to the change amount exceeds a predetermined threshold changing time (S3152c). The threshold change amount and the threshold changing time may be predetermined through various objects described above, and be stored in the memory 330. The threshold change amount and the threshold changing time may be a standard to determine whether the user's arm is accommodated in the space S for enough time. In a case in which the user's arm is accommodated in the space S for enough time, the change amount of the electrical value may exceed the threshold change amount. In a case in which the user's arm is accommodated in the space S for enough time, the changing time with respect to the change amount may exceed the threshold changing time.

For example, the control unit 300 may compare the change amount of the electrical value and the threshold change amount to determine whether the change amount of the electrical value exceeds the threshold change amount, and compare the changing time with respect to the change amount and the threshold changing time to determine whether the changing time exceeds the threshold changing time.

In at least one among the case in which the change amount exceeds the threshold change amount and the case in which the changing time exceeds the threshold changing time, the control unit 300 may determine that the user's arm is accommodated in the space S for enough time. In at least one among the case in which the change amount is less than the threshold change amount and the case in which the changing time is less than the threshold changing time, the control unit 300 may determine that the user's arm is not accommodated in the space S for enough time.

The control unit 300 may control the operation of the arm massage unit 500 based on at least one among the second determination result, the third determination result, and the fourth determination result (S3152d).

In at least one among the case in which the change amount exceeds the threshold change amount and the case in which the changing time exceeds the threshold changing time, and in a case in which the operation state is in the operating state, the control unit 300 may maintain the operation of the arm massage unit 500.

In at least one among the case in which the change amount is less than the threshold change amount and the case in which the changing time is less than the threshold changing time, and in a case in which the operation state is in the stop state, the control unit 300 may maintain the operation of the arm massage unit 500.

In at least one among the case in which the change amount is less than the threshold change amount and the case in which the changing time is less than the threshold changing time, and in a case in which the operation state is in the operating state, the control unit 300 may stop the arm massage unit 500 to change the operation of the arm massage unit 500.

In at least one among the case in which the change amount exceeds the threshold change amount and the case in which the changing time exceeds the threshold changing time, and in a case in which the operation state is in the stop state, the control unit 300 may operate the arm massage unit 500 to change the operation of the arm massage unit 500.

In addition, as described above, in a case in which the operation state of the arm massage unit 500 is changed into the operating state, the control unit 300 may recontrol the operation of the arm massage unit 500 (S3160). in the case in which the operation state of the arm massage unit 500 is changed into the operating state, the control unit 300 may control the operation of the arm massage unit 500 based on pneumatic pressure of the air cell 570.

For example, the control unit 300 may control the operation of the arm massage unit 500 based on whether pneumatic pressure of the air cell 570 exceeds the threshold pneumatic pressure (S3161).

For example, the control unit 300 may expand the air cell (S3161a). The control unit 300 may perform a fifth determination to determine whether pneumatic pressure of the expanded air cell exceeds the threshold pneumatic pressure (S3161b). The control unit 300 may control the operation of the arm massage unit 500 based on the fifth determination result (S3161c). Step S3161a is the same as step S3131a, step S3161b is the same as step S3131b, and step S3161c is the same as step S3131c. Accordingly, detailed description thereof will be omitted.

In another embodiment, the control unit 300 may control the operation of the arm massage unit 500 based on at least one among pneumatic pressure of the air cell 570, rechange amount of the electric value, and rechanging time with respect to the rechange amount (S3162).

For example, the control unit 300 may expand the air cell 570 (S3162a), and perform the fifth determination (S3162b). Step S3162a is the same as step S3161a, and step S3162b is the same as step S3161b. Accordingly, detailed description thereof will be omitted.

In a case in which pneumatic pressure of the expanded air cell 570 exceeds threshold pneumatic pressure, the control unit 300 may perform at least one among a sixth determination to determine whether the rechange amount of the electrical value changing after the expansion of the air cell 570 exceeds a predetermined threshold rechange amount and a seventh determination to determine whether the rechanging time with respect to the rechange amount exceeds a predetermined threshold rechanging time (S3162c).

The rechange amount of the electrical value may indicate the degree that the electrical value of the first bio-signal measuring electrode 980 is changed after the air cell 570 is expanded. The rechange amount may be determined based on at least one among a contact area between the user's arm and the first bio-signal measuring electrode 980 after the air cell 570 is expanded and the number of electrodes getting in contact with the user's arm after the air cell 570 is expanded. For example, the rechange amount may be greater when the contact area is large after the expansion of the air cell 570 than when the contact area is small after the expansion of the air cell 570. The rechange amount may be greater when the number of electrodes getting in contact with the user's arm is large after the expansion of the air cell 570 than when the number of electrodes getting in contact with the user's arm is small after the expansion of the air cell 570. In other words, the rechange amount may be increased as the contact area is large after the expansion of the air cell 570 or the number of the electrodes getting in contact with the user's arm is large after the expansion of the air cell 570.

The rechanging time with respect to the rechange amount may range from the moment the electrical value of the first bio-signal measuring electrode 980 starts to change after the expansion of the air cell 570 to the moment the electrical value of the first bio-signal measuring electrode 980 is not changed. The rechanging time may be determined based on contact time that the user's arm gets in contact with the first bio-signal measuring electrode 980 after the expansion of the air cell 570. For instance, the rechanging time may be longer when the contact time is long after the expansion of the air cell 570 than when the contact time is short after the expansion of the air cell 570. In other words, the rechanging time may be increased as the user's arm gets in contact with the first bio-signal measuring electrode 980 for a long period of time after the expansion of the air cell 570.

The threshold rechange amount and the threshold rechanging time may be predetermined through various objects described above, and be stored in the memory 330. The threshold rechange amount and the threshold rechanging time may be a standard to determine whether the user's arm is accommodated in the space S for enough time. In a case in which the user's arm is accommodated in the space S for enough time, the rechange amount of the electrical value may exceed the threshold rechange amount. In a case in which the user's arm is accommodated in the space S for enough time, the rechanging time with respect to the rechange amount may exceed the threshold rechanging time.

The threshold change amount and the threshold rechange amount may be the same or different, and the threshold changing time and the threshold rechanging time may be the same or different. Preferably, the threshold rechange amount may be greater than the threshold change amount, but is not limited thereto. For instance, the threshold rechange amount may be smaller than the threshold change amount, but is not limited thereto. For instance, the threshold rechanging time may be longer than the threshold changing time.

For example, the control unit 300 may compare the rechange amount of the electrical value and the threshold rechange amount to determine whether the rechange amount exceeds the threshold rechange amount. The control unit 300 may compare the rechanging time with respect to the rechange amount and the threshold rechanging time to determine whether the rechanging time exceeds the threshold rechanging time.

In at least one among a case in which the rechange amount exceeds the threshold rechange amount, the control unit 300 and a case in which the rechanging time exceeds the threshold rechanging time, the control unit 300 may determine that the user's arm is exactly accommodated in the space S for enough time. In at least one among a case in which the rechange amount is less than the threshold rechange amount, the control unit 300 and a case in which the rechanging time is less than the threshold rechanging time, the control unit 300 may determine that the user's arm is not exactly accommodated in the space S for enough time.

The control unit 300 may control the operation of the arm massage unit 500 based on at least one among the sixth determination result and the seventh determination result (S3162d). In at least one among a case in which the rechange amount exceeds the threshold rechange amount, the control unit 300 and a case in which the rechanging time exceeds the threshold rechanging time, the control unit 300 may maintain the operation of the arm massage unit 500.

In at least one among a case in which the rechange amount is less than the threshold rechange amount, the control unit 300 and a case in which the rechanging time is less than the threshold rechanging time, the control unit 300 may stop the arm massage unit 500 to change the operation of the arm massage unit 500.

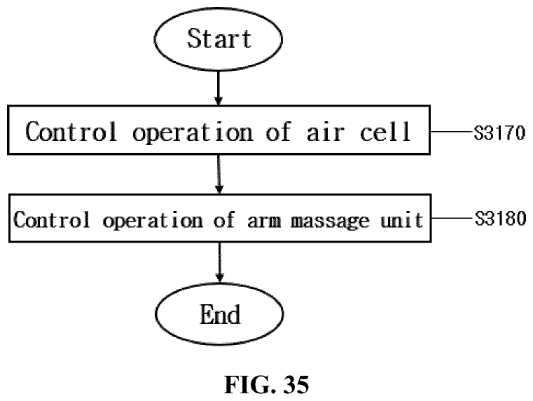

FIG. 35 is a view for depicting an arm massage unit control operation of the control unit according to an embodiment of the present disclosure.

As described above with reference to FIGS. 33 and 34, the control unit 300 may control the operation of the first bio-signal measuring unit 550 in the sensing section, and control the operation of the arm massage unit 500 based on the electrical value of the first bio-signal measuring electrode 980 in the operating section, but is not limited thereto.

For example, the control unit 300 may control the operation of the air cell 570 in the sensing section (S3170), and control the operation of the arm massage unit 500 based on pneumatic pressure of the air cell 570 (S3180).

First, the control unit 300 may expand the air cell 570 in the sensing section (S3170). Since step S3170 is the same as step S3131a, detailed description thereof will be omitted.

Thereafter, the control unit 300 may control the operation of the arm massage unit 500 in the operating section based on pneumatic pressure of the expanded air cell 570 (S3180).

Hereinafter, step S3180 will be described in detail.

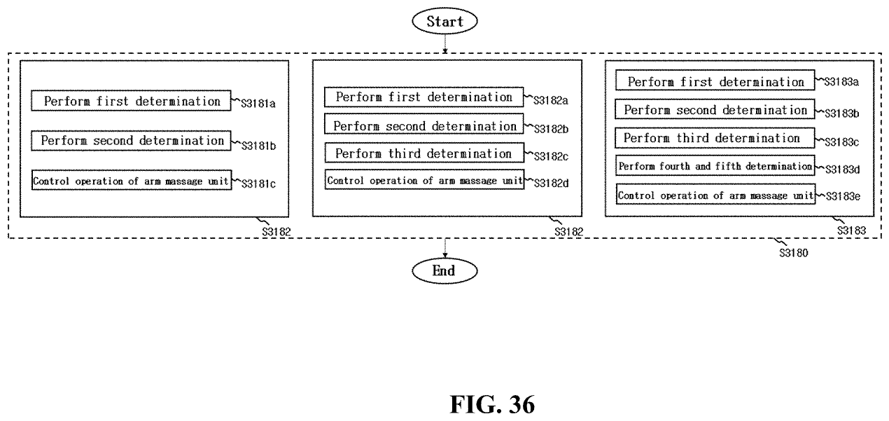

FIG. 36 is a view for depicting the arm massage unit control operation illustrated in FIG. 35.

According to an embodiment of the present disclosure, the control unit 300 may control the operation of the arm massage unit 500 based on the pneumatic pressure of the expanded air cell 570 and the operation state of the arm massage unit 500 (S3180).

For example, the control unit 300 may determine whether the pneumatic pressure of the expanded air cell 570 exceeds a threshold pneumatic pressure, and control the operation of the arm massage unit 500 based on the determination result and the operation state of the arm massage unit 500 (S3181).

For instance, the control unit 300 may perform the first determination to determine whether the pneumatic pressure of the expanded air cell 570 exceeds the threshold pneumatic pressure (S3181a). Since step S3181a is the same as step S3161b, detailed description thereof will be omitted.

The control unit 300 may perform the second determination to determine whether the arm massage unit 500 is in the operating state (S3181b). Since step S3181b is the same as step S3121b, detailed description thereof will be omitted.

The control unit 300 may control the operation of the arm massage unit 500 based on the first determination result and the second determination result (S3181c).

In at least one among a case in which the pneumatic pressure of the expanded air cell 570 is less than the threshold pneumatic pressure and the operation state is in the stop state and a case in which the pneumatic pressure of the expanded air cell 570 exceeds the threshold pneumatic pressure and the operation state is in the operating state, the control unit 300 may maintain the operation of the arm massage unit 500.

In a case in which the pneumatic pressure of the expanded air cell 570 is less than the threshold pneumatic pressure and the operation state is in the operating state, the control unit 300 may stop the arm massage unit 500 to change the operation of the arm massage unit 500.

In a case in which the pneumatic pressure of the expanded air cell 570 exceeds the threshold pneumatic pressure and the operation state is in the stop state, the control unit 300 may operate the arm massage unit 500 to change the operation of the arm massage unit 500.

In another embodiment, the control unit 300 may control the operation of the arm massage unit 500 based on the pneumatic pressure of the expanded air cell 570, the operation state of the arm massage unit 500, and the electrical value of the first bio-signal measuring electrode 980 (S3182).

For example, the control unit 300 may perform the first determination to determine whether the pneumatic pressure of the expanded air cell 570 exceeds the threshold pneumatic pressure (S3181a), and perform the second determination to determine whether the operation state is in the operating state (S3182b). Since step S3182a is the same as step S3181a and step S3182b is the same as step S3181b, detailed description thereof will be omitted.

In a case in which the pneumatic pressure of the expanded air cell 570 exceeds the threshold pneumatic pressure, the control unit 300 may perform the third determination to determine whether the electrical value of the first bio-signal measuring electrode 980 is changed (S3182c). Since step S3182a is the same as step S3181a and step S3182c is the same as step S3151a, detailed description thereof will be omitted.

The control unit 300 may control the operation of the arm massage unit 500 based on the second determination result and the third determination result (S3182d).

In at least one among a case in which the electrical value is not changed and the operation state is in the stop state and a case in which the electrical value is changed and the operation state is in the operating state, the control unit 300 may maintain the operation of the arm massage unit 500.

In a case in which the electrical value is not changed and the operation state is in the operating state, the control unit 300 may stop the arm massage unit 500 to change the operation of the arm massage unit 500.

In a case in which the electrical value is changed and the operation state is in the stop state, the control unit 300 may operate the arm massage unit 500 to change the operation of the arm massage unit 500.

In another embodiment, the control unit 300 may change the operation of the arm massage unit 500 based on the pneumatic pressure of the expanded air cell 570, the operation state of the arm massage unit 500, and the change amount and the changing time of the electrical value of the first bio-signal measuring electrode 980 (S3183).

For instance, the control unit 300 may perform the first determination to determine whether the pneumatic pressure of the expanded air cell 570 exceeds the threshold pneumatic pressure (S3183a), perform the second determination to determine whether the operation state is in the operating state (S3183b), and perform the third determination to determine whether the electrical value is changed (S3183c). Since step S3183a is the same as step S3182a, step S3183b is the same as step S3182b and step S3183c is the same as step S3182c, detailed description thereof will be omitted.

In a case in which the electrical value is changed, the control unit 300 may perform at least one among the fourth determination to determine whether the change amount of the electrical value exceeds the threshold change amount and the fifth determination to determine whether the changing time with respect to the change amount exceeds the threshold changing time (S3183d). Since step S3183d is the same as step S3152c, detailed description thereof will be omitted.

The control unit 300 may control the operation of the arm massage unit 500 based on at least one among the second determination result, the fourth determination result, and the fifth determination result (S3183e). Since step S3183e is the same as step S3152d, detailed description thereof will be omitted.

As described above with reference to FIGS. 31 to 36, the control unit 300 may sense the user's arm to control the operation of the arm massage unit 500, thereby preventing waste of power of the arm massage unit 500 and preventing user satisfaction with the massage apparatus 100 from lowering.

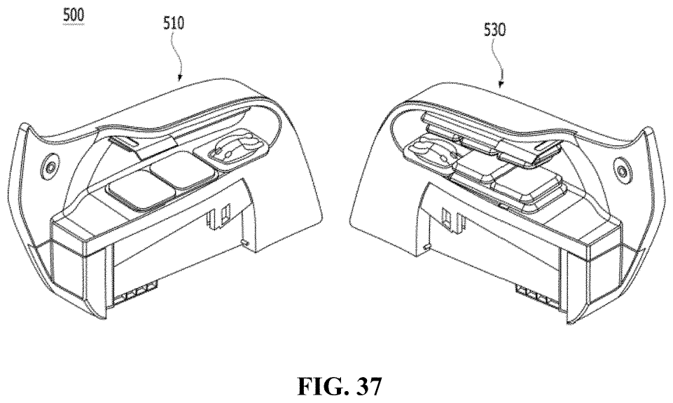

FIG. 37 is a view for depicting the operation of the arm massage unit and the operation of the body massage unit according to an embodiment of the present disclosure.

According to an embodiment of the present disclosure, the control unit 300 may independently control the operations of the arm massage units 510 and 530 based on sensing signals of the sensing units 591 and 593.

The control unit 300 may control the operation of the first arm massage unit 510 based on a first sensing signal of the first sensing unit 591 and a first operation state of the first arm massage unit 510. The first operation state may be at least one among a first operating state in which the first arm massage unit 510 is operating and a first stop state in which the first arm massage unit 510 is stopped.

For instance, the control unit 300 may perform the first determination to determine whether the first sensing signal is received. Since the first determination is the same as the operation to determine whether the sensing signal is received, detailed description thereof will be omitted.

The control unit 300 may perform the second determination to determine whether the first operation state of the first arm massage unit 510 is in the first operating state. Since the second determination is the same as the operation to determine whether the operation state is in the operating state, detailed description thereof will be omitted.

The control unit 300 may control the operation of the first arm massage unit 510 based on the first determination result and the second determination result.

In at least one among a case in which the first sensing signal is not received and the first operation state is in the first stop state and a case in which the first sensing signal is received and the first operation state is in the first operating state, the control unit 300 may maintain the operation of the first arm massage unit 510.

In a case in which the first sensing signal is not received and the first operation state is in the first operating state, the control unit 300 may stop the first arm massage unit 510 to change the operation of the first arm massage unit 510.

In a case in which the first sensing signal is received and the first operation state is in the first stop state, the control unit 300 may operate the first arm massage unit 510 to change the operation of the first arm massage unit 510.

In addition, the control unit 300 may control the operation of the second arm massage unit 530 based on a second sensing signal of the second sensing unit 593 and a second operation state of the second arm massage unit 530. The second operation state may be at least one among a second operating state in which the second arm massage unit 530 is operating and a second stop state in which the second arm massage unit 530 is stopped.

For instance, the control unit 300 may perform the third determination to determine whether the second sensing signal is received. Since the third determination is the same as the operation to determine whether the sensing signal is received, detailed description thereof will be omitted.

The control unit 300 may perform the fourth determination to determine whether the second operation state of the second arm massage unit 530 is in the second operating state. Since the fourth determination is the same as the operation to determine whether the operation state is in the operating state, detailed description thereof will be omitted.

The control unit 300 may control the operation of the second arm massage unit 530 based on the third determination result and the fourth determination result.

In at least one among a case in which the second sensing signal is not received and the second operation state is in the second stop state and a case in which the second sensing signal is received and the second operation state is in the second operating state, the control unit 300 may maintain the operation of the second arm massage unit 530.

In a case in which the second sensing signal is not received and the second operation state is in the second operating state, the control unit 300 may stop the second arm massage unit 530 to change the operation of the second arm massage unit 530.

In a case in which the second sensing signal is received and the second operation state is in the second stop state, the control unit 300 may operate the second arm massage unit 530 to change the operation of the second arm massage unit 530.

As described above, the control unit 300 controls the operation of the first arm massage unit 510 based on the first determination result and the second determination result and controls the operation of the second arm massage unit 530 based on the third determination result and the fourth determination result, but is not limited thereto. For example, the control unit 300 may control the operation of each arm massage unit based on the determination results to which the contents described with reference to FIGS. 31 to 36 are applied.

Accordingly, the control unit 300 may independently control the operation of each arm massage unit by sensing each arm and controlling the operation of each arm massage unit.

According to an embodiment of the present disclosure, the control unit 300 may control the operation of each arm massage unit as described above in a state in which the body massage unit 2100 operates or does not operate. In other words, the control unit 300 may independently control the operations of the arm massage units 510 and 530 and the operation of the body massage unit 2100.

According to an embodiment of the present disclosure, the control unit 300 may control the operation of the body massage unit 2100 in a state in which each arm massage unit operates or does not operate.

For example, the control unit 300 may control the body massage unit 2100 based on a control signal with respect to the body massage unit 2100. In this instance, the control signal may be at least one among an operation signal (or driving signal) for operating the massage unit 2100 and a stop signal for stopping the body massage unit 2100. The control signal may be generated based on a user's input.

The input unit 350 may generate a control signal based on the user's input and transmit the control signal to the control unit 300. In this instance, the user's input may be at least one among an operation input for operating the body massage unit 2100, a stop input for stopping the body massage unit 2100, and a change input for changing the operation of the body massage unit 2100. The control unit 300 may operate or stop the body massage unit 2100 based on the control signal.

In another embodiment, the control unit 300 may control the operation of the body massage unit 2100 based on the first and third determination results described with reference to FIG. 37, and a third operation state of the body massage unit 2100. In this instance, the third operation state may be at least one among a third operating state in which the body massage unit 2100 is operated and a third stop state in which the body massage unit 2100 is stopped.

For instance, the control unit 300 may perform the fifth determination to determine whether at least one among the first sensing signal and the second sensing signal is received after performing the first determination and the third determination.

The control unit 300 may perform the sixth determination to determine whether the third operation state is in the third operating state.

The control unit 300 may control the operation of the body massage unit 2100 based on the fifth determination result and the sixth determination result.

In at least one among a case in which at least one among the first sensing signal and the second sensing signal is received and the third operation state is in the third operating state and a case in which both of the first sensing signal and the second sensing signal are not received and the third operation state is in the third stop state, the control unit 300 may maintain the operation of the body massage unit 2100.

In a case in which at least one among the first sensing signal and the second sensing signal is received and the third operation state is in the third stop state, the control unit 300 may operate the body massage unit 2100 to change the operation of the body massage unit 2100.

In a case in which both of the first sensing signal and the second sensing signal are not received and the third operation state is in the third operating state, the control unit 300 may stop the body massage unit 2100 to change the operation of the body massage unit 2100.

As described above, the control unit 300 controls the operation of the body massage unit 2100 based on the first determination result, the third determination result and the third operation state, but is not limited thereto. For example, the control unit 300 may control the operation of the body massage unit 2100 based on the third operation state and the determination results to which the contents described with reference to FIGS. 31 to 36 are applied.

Various embodiments according to the present disclosure have been described. It will be understood by those skilled in the art that the present disclosure may be implemented in a modified form without departing from the essential characteristics of the present disclosure. Therefore, the disclosed embodiments should be considered in the expository perspective rather than limitative perspective. Therefore, the scope of the present disclosure is defined not by the detailed description of the invention but by the appended claims, and all differences within the equivalent scope will be construed as being included in the present disclosure.

Meanwhile, embodiments of the present disclosure described above may be made as a computer-executable program, and may be implemented in a general-purpose digital computer that operates the program using a computer-readable recording medium. The computer-readable recording medium includes a storage medium, such as a magnetic storage medium (e.g., a ROM, a floppy disk, a hard disk, etc.), or an optical recording medium (e.g., a CD-ROM, a DVD, etc.).

### CROSS-REFERENCE TO RELATED APPLICATIONS

The present application claims priority to Korean patent application Nos. 10-2021-0169550 filed on Nov. 30, 2020, and 10-2022-0006099 filed on Jan. 14, 2022, the entire contents of which are hereby incorporated by reference.

What is claimed is:

1. A massage apparatus comprising:
an arm massage unit for massaging a user's arm; and
a control unit for controlling the arm massage unit,
wherein the arm massage unit comprises a space and an electrode arranged in the space, and
wherein the control unit determines whether an electrical value of the electrode is changed, and controls the operation of the arm massage unit based on a determination result and an operation state of the arm massage unit.

2. The massage apparatus according to claim 1, wherein the electrical value is at least one among voltage, current, resistance, and impedance of the electrode, and
wherein the operation state is at least one among an operating state in which the arm massage unit is operated and a stop state in which the arm massage unit is stopped.

3. The massage apparatus according to claim 2, wherein the control unit performs a first determination to determine whether the electrical value is changed, performs a second determination to determine whether the operation state is in the operating state, and controls the operation of the arm massage unit based on the first determination result and the second determination result.

4. The massage apparatus according to claim 3, wherein in at least one among a case in which the electrical value is not changed and the operation state is in the stop state and a case in which the electrical value is changed and the operation state is in the operating state, the control unit maintains the operation of the arm massage unit,
wherein in a case in which the electrical value is not changed and the operation state is in the operating state, the control unit stops the arm massage unit to change the operation of the arm massage unit, and
wherein in a case in which the electrical value is changed and the operation state is in the stop state, the control unit operates the arm massage unit to change the operation of the arm massage unit.

5. The massage apparatus according to claim 3, wherein in a case in which the electrical value is changed, the control unit performs at least one among a third determination to determine whether a change amount of the electrical value exceeds a predetermined threshold change amount and a fourth determination to determine whether changing time with respect to the change amount exceeds a predetermined threshold changing time, and controls the operation of the arm massage unit based on the second determination result and at least one among the third determination result and the fourth determination result.

6. The massage apparatus according to claim 3, wherein in at least one among the case in which the change amount exceeds the threshold change amount and the case in which the changing time exceeds the threshold changing time, and in a case in which the operation state is in the operating state, the control unit maintains the operation of the arm massage unit,
wherein in at least one among the case in which the change amount is less than the threshold change amount and the case in which the changing time is less than the threshold changing time, and in a case in which the operation state is in the stop state, the control unit maintains the operation of the arm massage unit,
wherein in at least one among the case in which the change amount is less than the threshold change amount and the case in which the changing time is less than the threshold changing time, and in a case in which the operation state is in the operating state, the control unit stops the arm massage unit to change the operation of the arm massage unit, and
wherein in at least one among the case in which the change amount exceeds the threshold change amount and the case in which the changing time exceeds the threshold changing time, and in a case in which the operation state is in the stop state, the control unit operates the arm massage unit to change the operation of the arm massage unit.

7. The massage apparatus according to claim 3, wherein the arm massage unit comprises an air cell disposed in the space, and
wherein in a case in which the operation state of the arm massage unit is changed into the operating state, the control unit controls the operation of the arm massage unit based on pneumatic pressure of the air cell.

8. The massage apparatus according to claim 7, wherein the control unit expands the air cell, performs a fifth determination to determine whether the pneumatic pressure of the expanded air cell exceeds a predetermined threshold pneumatic pressure, and controls the operation of the arm massage unit based on the fifth determination result.

9. The massage apparatus according to claim 8, wherein in a case in which the pneumatic pressure of the expanded air cell exceeds the threshold pneumatic pressure, the control unit maintains the operation of the arm massage unit, and
wherein in a case in which the pneumatic pressure of the expanded air cell is less than the threshold pneumatic pressure, the control unit stops the arm massage unit to change the operation of the arm massage unit.

10. The massage apparatus according to claim 8, wherein in a case in which the pneumatic pressure of the expanded air cell exceeds the threshold pneumatic pressure, the control unit performs at least one among a sixth determination to determine whether a rechange amount of the electrical value changing after the expansion of the air cell exceeds a predetermined threshold rechange amount and a seventh determination to determine whether rechanging time with respect to the rechange amount exceeds a predetermined threshold rechanging time, and controls the operation of the arm massage unit based on at least one among the sixth determination result and the seventh determination result.

11. The massage apparatus according to claim 10, wherein the threshold change amount and the threshold rechange amount are different from each other, and the threshold changing time and the threshold rechanging time are different from each other.

12. The massage apparatus according to claim 10, wherein in at least one among a case in which the rechange amount exceeds the threshold rechange amount and a case in which the rechanging time exceeds the threshold rechanging time, the control unit maintains the operation of the arm massage unit, and
wherein in at least one among a case in which the rechange amount is less than the threshold rechange amount and a case in which the rechanging time is less than the threshold rechanging time, the control unit stops the arm massage unit to change the operation of the arm massage unit.

13. The massage apparatus according to claim 1, wherein the electrode measures biometric information of the user.
